(19) United States
(12) Patent Application Publication
(10) Pub. No.: US 2021/0283064 A1
(43) Pub. Date: Sep. 16, 2021

(54) STABLE, BIOADHESIVE, AND DIFFUSION-RESTRICTIVE COACERVATE

(71) Applicant: The Chinese University of Hong Kong, Shatin (CN)

(72) Inventors: Liming BIAN, Tai Wai (CN); Pengchao Zhao, Shatin (CN); Xiayi Xu, Shatin (CN)

(21) Appl. No.: 17/185,269

(22) Filed: Feb. 25, 2021

Related U.S. Application Data
(60) Provisional application No. 62/981,310, filed on Feb. 25, 2020.

(51) Int. Cl. A61K 9/51; A61K 9/08; A61K 45/06; A61K 9/06

(52) U.S. Cl. CPC A61K 9/5146; A61K 9/08; B82Y 5/00; A61K 9/5115; A61K 9/06; A61K 45/06

(57) ABSTRACT

Provided herein are nanoparticle assembled (NPA) coacervates, the nanoparticles of which each include a hydrophobic core and a plurality of hydrophilic polymeric chains extending from the hydrophobic core. The hydrophilic chains include functional end groups capable of non-covalent interactions with one another upon assembly of the nanoparticles into the coacervates. Also provided are methods for forming the coacervates, reversibly switching the physiological states of the coacervates, transiently activating macromolecular uptake by the coacervates, and administering the coacervates to a subject.

### CROSS-REFERENCE TO RELATED APPLICATIONS

The present patent application claims the benefit of U.S. Provisional Patent Application No. 62/981,310, filed Feb. 25, 2020, the contents of which are incorporated by reference herein for all purposes.

### BACKGROUND

Three-dimensional (3D) culture of cells in designer biomaterial matrices provides a biomimetic cellular microenvironment and can yield critical insights into cellular behaviors not available from conventional two-dimensional culture on tissue culture plastics (Wade & Burdick, 15 Mater. Today 454 (2012); Hussey, Dziki, & Badylak, 3 Nat. Rev. Mater. 159 (2018)). Hydrogels are widely used as a 3D polymeric matrix supporting 3D cell culture (Rosales & Anseth, 1 Nat. Rev. Mater. 105012 (2016); Lutolf, 8 Nat. Mater. 451 (2009); Seliktar, 336 Science 1124 (2012); Zhang & Khademhosseini, 356 Science eaaf3627 (2017)). However, the highly hydrated and permeable network of hydrogels cannot establish the concentrated macromolecular spatial heterogeneity that is typically found inside and outside of cells. Intracellular space in particular is highly crowded with macromolecules and contains diverse membrane-less liquid sub-compartments, which are essentially liquid phase-separated coacervates (FIG. 1). Meanwhile, extracellular coacervates, such as those found in neural synaptic junctions, are also crucial to cellular functions such as neuronal communication (Zeng et al., 166 Cell 1163 (2016); Feng, Chen, Zeng, & Zhang, 57 Curr. Opin. Neurobiol. 1 (2019)). These coacervate compartments can develop heterogeneous multiphase architectures such as multilayered core-shell substructures with distinct liquid phases (Feric et al., 165 Cell 1686 (2016); Jain et al., 164 Cell 487 (2016)), and vacuolated morphologies (Schmidt & Rohatgi, 16 Cell reports 1228 (2016); Kistler et al., 7 elife e37949 (2018)), thus potentially localizing and segregating distinct sets of biological processes without relying on membrane boundaries to regulate cellular function (Shin & Brangwynne, 357 Science eaaf4382 (2017)). Therefore, developing synthetic coacervates in vitro can not only provide novel insights into the complexity of naturally occurring coacervates but also facilitate the exploration of potential applications of coacervates, such as customizing a segregated macromolecular environment to tune cellular behaviors in a spatiotemporally controlled manner.

Extensive prior works have demonstrated the in vitro preparation of synthetic complex coacervates via the electrostatically driven fluid-fluid phase separation of complexed polyelectrolytes with opposite charges (FIG. 2) (Koga, Williams, Perriman, & Mann, 3 Nat. Chem. 720 (2011); Aumiller & Keating, 8 Nat. Chem. 129 (2016); Mandla, Davenport, Huyer, & Radisic, 2 APL bioengineering 021503 (2018); Martin et al., 58 Angew. Chem. Int. Ed. 14594 (2019); Lu & Spruijt, 142 J. Am. Chem. Soc. 2905 (2020)). These complex coacervates that usually exhibit homogeneous morphologies can act as simplified abiogenic phase-separated systems to encapsulate diverse macromolecules (Jeon, Wolfson, & Alsberg, 27 Adv. Mater. 2216 (2015); McTigue & Perry, 15 Soft matter 3089 (2019); Blocher, McTigue, & Perry, Small 1907671 (2020)), and enhance biochemical reactions (Love et al., 59 Angew. Chem. Int. Ed. 5950 (2020); Drobot et al., 9 Nat. Commun. 1 (2018); Gobbo et al., 11 Nat. Commun. 1 (2020)). However, the macromolecular concentrates in such synthetic coacervates inevitably undergo rapid diffusional exchange with the surrounding dilute solution, thus failing to isolate the loaded macromolecules within the coacervate phase for even a few hours (Jia, Hentrich, & Szostak, 44 Orig. Life Evol. Biosph. 1 (2014); Aumiller Jr., Cakmak, Davis, & Keating, 32 Langmuir 10042 (2016); Wei et al., 9 Nat. Chem. 1118 (2017); Long, Johnson, Jeffries, Hara, & Wang, J. Control. Release 73 (2017)). Beyond the simple homogeneous morphology of synthetic complex coacervates described above, coacervates with heterogeneous multiphase architectures such as vacuolated coacervates may provide macromolecular diffusion barriers and segregate macromolecules into the enclosed internal vacuoles, thus better controlling the spatiotemporal localization of macromolecules. Because the vacuoles in coacervates tend to coalesce upon contact or be directly excluded from the liquid coacervates (Yin et al., 7 Nat. Commun. 10658 (2016); Banerjee, Milin, Moosa, Onuchic, & Deniz, 129 Angew. Chem. 11512 (2017)), few studies have demonstrated synthetic complex coacervates with metastable vacuoles which can function as a stable cell 3D microenvironment under the physiological conditions.

### BRIEF SUMMARY

The present disclosure generally relates to nanoparticle-assembled (NPA) coacervates, e.g., vacuolated nanoparticle-assembled coacervates, that, when employed, provide several advantageous improvements. For example, it is beneficial for coacervate compartments, e.g., coacervate microdroplets or vacuoles within coacervates, to have high size uniformity and long-term stability with minimal coalescence under physiological conditions. It is also beneficial for these coacervate compartments to restrict the diffusional exchange of macromolecules with the surrounding liquid phase so as to establish the spatiotemporal macromolecular heterogeneity and precisely control the behaviors of cells encapsulated in the vacuoles. It can also be useful for the macromolecular diffusion barrier property of the coacervate compartments to be capable of being reversibly switched on and off by an external stimulus. Conventional approaches to the formation of synthetic coacervates through complexation of oppositely-charged electrolytes generally do not create coacervates possessing these and other important attributes.

To address the above challenges, the inventors have now discovered a different approach to fabricate vacuolated coacervates or coacervate microdroplets via non-covalent-bonding-driven in-situ self-assembly of core-shell nanoparticles having diverse compositions. The vacuole or microdroplet compartments of nanoparticle-assembled coacervates exhibit excellent anti-coalescence under physiological conditions (FIG. 2). The stable NPA coacervate compartments further demonstrate significantly low size polydispersity compared with that of conventional complex coacervate compartments. Furthermore, the provided NPA coacervates can segragate macromolecules into vacuole or microdroplet compartments via mechanical agitation, while also restricting macromolecular exchange with the outside liquid phase under resting condition for several days. Upon transition to a hydrogel state, however, this restriction on macromolecular diffusion is nearly immediately abolished. Importantly, the NPA coacervates can thus form liquid 3D microcompartments with controlled macromolecular spatiotemporal distribution heterogeneity to modulate cellular behaviors, and therefore can regulate diverse functions of cells encapsulated in such macromolecule concentrates.

In one aspect, the disclosure is to a population of coacervates, each including an assembly of nanoparticles. Each nanoparticle includes a hydrophobic core and a plurality of hydrophilic chains extending from the hydrophobic core. The hydrophilic chains each include a functional end group. The coacervates further include non-covalent interactions between at least a portion of the functional end groups. In a related aspect, the invention provides a composition comprising a population of the coacervates as described above and herein with a physiologically acceptable excipient.

In another aspect, the disclosure is to a method of forming a population of coacervates. The method includes providing a population of polymers. Each polymer includes hydrophilic polymeric chains. Each hydrophilic polymeric chain includes a functional end group. The method further includes fabricating, via self-assembly of the polymers, a population of nanoparticles. The fabricated nanoparticles each include a hydrophobic core and a plurality of the hydrophilic chains extending from the hydrophobic core. The method further includes forming, via non-covalent interactions between at least a portion of the functional end groups, the population of coacervates.

In another aspect, the disclosure is to a method of reversibly switching the physiological state of a population of coacervates. The method includes providing a population of coacervates as disclosed herein, wherein the coacervates have a first physiological state. The method further includes changing the temperature of the coacervates beyond an upper critical solution temperature, thereby switching the physiological state of the coacervates from the first physiological state to a second physiological state.

In another aspect, the disclosure is to a method of transitioning a population of coacervates from a compartmentalized state, e.g., a vacuolated liquid or microdroplet state, to a hydrogel state. The method include providing a population of coacervates as disclosed herein, wherein the coacervates have a vacuolated liquid state. The method further includes contacting the coacervates with Ti⁴⁺ titanium ions, thereby transitioning the coacervates to a hydrogel state.

In another aspect, the disclosure is to a method of transiently activating uptake of a macromolecule by a population of coacervates. The method includes providing a population of coacervates as disclosed herein. The method further includes agitating the coacervates in a buffer including the macromolecule, thereby increasing the uptake efficiency of the coacervates and activating uptake of the macromolecule by the coacervates. The method further includes stopping agitation of the coacervates, thereby increasing the barrier efficiency of the coacervates.

In another aspect, the disclosure is to a method of adhering a population of coacervates within the body of a subject. The method includes providing a population of coacervates as disclosed herein, wherein the functional end groups include catechol. The method further includes administering the coacervates to the subject, thereby adhering the coacervates within the body of the subject.

In another aspect, the disclosure is to a method of delivering a compound to a subject in need thereof. The method includes providing a population of coacervates as disclosed herein, wherein the coacervates encapsulate a therapeutically effective amount of the compound. The method further includes administering the coacervates to the subject, thereby delivering the compound.

In another aspect, the disclosure is to a method of treating an inflammatory bowel disease. The method includes providing a population of coacervates as disclosed herein, wherein the coacervates encapsulate a therapeutically effective amount of a compound for treating the inflammatory bowel disease, and wherein the function end groups comprise catechol. The method further includes administering the coacervates to the subject, thereby adhering at least a portion of the coacervates to the subject and delivering the compound.

### BRIEF DESCRIPTION OF THE DRAWINGS

The disclosure is described in detail below with reference to the appended drawings.

FIG. 1 is an illustration of membraneless coacervates found inside and outside of living cells that frequently exhibit multiphase substructures, such as multilayered nucleoli and vacuolated germ granules.

FIG. 2 is an illustration of the formation of homogeneous complex coacervate though a conventional method of complexation of polymer species, versus formation vacuolated coacervate through the provided method of self-assembly of polymeric nanoparticles.

FIG. 3 is an illustration of formation of core-shell nanoparticles displaying surface catechol groups.

FIG. 4 is a graph of DLS analysis results confirming the successful synthesis of as-prepared core-shell nanoparticles.

FIG. 5 is an illustration of dialysis of core-shell nanoparticles bearing surface catechol groups against deionized water under room temperature for 24 hours, thereby inducing self-assembly of core-shell nanoparticles and yielding the dense phase nanoparticle-assembled (NPA) coacervate through fluid-fluid phase separation.

FIG. 6 is an illustration of the failure of negative control nanoparticles without surface catechol groups to form coacervates.

FIG. 7 is a graph of the thermo-responsive rheological properties of the NPA coacervate.

FIG. 8 presents photographs showing reversible formation of vacuolated NPA coacervate by tuning temperature. Scale bar: 100 μm.

FIG. 9 is an illustration of the diameter change of vacuoles within NPA coacervate over time under physiological buffer conditions.

FIG. 10 is a graph of the different paths of diameter change of vacuoles within NPA coacervate over time in acidic or physiological buffer condition at 37° C.

FIG. 11 is a graph showing that the stabilized vacuoles within NPA coacervate of FIG. 10 at 210 minutes (pH 7.4) had an average diameter of 46.2 μm with low size polydispersity.

FIG. 12 presents photographs showing anti-coalescence properties of the provided NPA coacervate. Scale bar: 50 μm.

FIG. 13 presents TEM and SAXS data of the structure of vacuolated NPA coacervate comprising the self-assembled core-shell nanoparticles. Scale bar: 100 nm.

FIG. 14 illustrates the mechanism of the anti-coalescence property of vacuolated NPA coacervate, including the diminishing hydrogen bonding and the increasing covalent crosslinking density between surface catechol groups due to catechol—quinone oxidation under physiological condition.

FIG. 15 illustrates differential macromolecular uptake into vacuoles within the NPA coacervate matrix under resting and mechanically-agitated conditions. The NPA coacervate matrix can be transiently disrupted by brief mechanical agitation to load macromolecules into inner vacuoles before fully recovering the original vacuolated structure. Mechanical agitation induces macromolecular uptake into the internal vacuoles, while the NPA coacervate matrix limits macromolecular diffusion.

FIG. 16 presents fluorescence images showing the distribution of different proteins with different pI in the NPA coacervate, and graphs showing the corresponding fluorescence intensity profiles along the dash lines drawn across the vacuoles. Scale bar: 50 μm.

FIG. 17 presents confocal fluorescence and microscopy images showing segregation of non-proteinaceous macromolecules in the vacuolated NPA coacervate. 3D reconstruction of the confocal images confirmed that the vacuoles were fully enclosed in NPA (H₁) coacervate matrix labeled by red fluorescence (xy and yz plane). The microscopy images show the distribution of pre-loaded dextran-FITC of different molecular weights in the NPA (H₁) coacervate after 1 day. Scale bar: 50 μm.

FIG. 18 presents a fluorescence image of the three-dimensional spatial distribution of BSA among a population of the provided coacervate compartments.

FIG. 19 presents confocal fluorescence images and corresponding intensity profiles confirming that under resting state the diffusion of BSA from both dilute solution (BSA-Texas red) and vacuoles (BSA-FITC) to the coacervate matrix was limited. The infiltration of BSA-Texas red from dilute solution across the NPA coacervate matrix into vacuoles was even more limited. The ratio between the mean fluorescent intensity (MFI) from the infiltrated BSA-Texas red in the coacervate matrix (C) and vacuoles (V) was quantified as the barrier efficiency (BE), where BE=[MFI in the coacervate (IC)]/[MFI in the vacuoles (IV)]. The barrier efficiency (BE) showed that the concentration of infiltrated BSA-Texas Red in the NPA (H1) coacervate matrix was more t0han 28 times that of the vacuole interior on day 1.

FIG. 20 is a graph showing no significant difference between the encapsulation efficiencies of diverse dextran-FITCs and BSA.

FIG. 21 presents graphs showing the fraction of released dextran detected outside the NPA coacervates, and the fraction of released BSA pre-loaded in the vacuoles of NPA 1coacervates, PEG diacrylate hydrogel, and polyacrylamide (PAAM) hydrogel after 1 day.

FIG. 22 illustrates the replacement of the hydrophobic cores of core-shell nanoparticles with the octadecyl (C18) group.

FIG. 23 is a graph of the rheological behavior of NPA (H₂) coacervates demonstrating that the coacervates exhibit hydrogel-coacervate transition behavior.

FIG. 24 presents images showing that dextran-FITC with varied molecular weights (10, 20, and 40 kDa) can be successfully loaded and segregated in the internal vacuoles of NPA (H₂) coacervate after incubation in 1× PBS at 37° C. on day 1.

FIG. 25 illustrates the replacement of the hydrophobic cores of core-shell nanoparticles with the thermo-responsive H₃ (PNIPAM) hydrophobic core.

FIG. 26 presents images showing that dextran-FITC (10 kDa) can be successfully loaded and segregated in the internal vacuoles of NPA (H₃) coacervate after incubation in 1× PBS at 37° C.

FIG. 27 presents an illustration and images showing the death of Hela cells pre-suspended in PBS during encapsulation despite 1-day culture in FBS medium, because of a lack of preloaded nutrients and the restricted influx of medium FBS into coacervate during culture. Scale bar: 25 μm.

FIG. 28 presents an illustration and images showing the viability of Hela cells pre-suspended in FBS medium during encapsulation, because of preloaded FBS in the coacervate during cell encapsulation. Scale bar: 25 μm.

FIG. 29 presents a photograph showing Hela cells well-encapsulated in vacuolated NPA coacervate after gentle mixing of the cell suspension. Scale bar: 100 μm.

FIG. 30 is an illustration of the temporal modulation of the pluripotency of ESCs by creating an LIF barrier at a resting state, or an LIF enrichment environment at a mechanically-agitated state.

FIG. 31 presents immunofluorescence staining images confirming the difference of pluripotent states of mouse ESCs, thus indicating that the outer layer NPA coacervate constructed a perfect barrier for supplemented LIF in culture medium and maintained the pluripotency of ESCs by segregating LIF into vacuoles under mechanical agitation. Scale bar: 20 μm.

FIG. 32 is a graph of the rheological properties of the NPA coacervates of FIG. 28 under 5 days of cell culture conditions.

FIG. 33 is a graph of qRT-PCR results confirming the difference of pluripotent states of mouse ESCs, thus indicating that the vacuolated NPA coacervate constructed a barrier for LIF at resting condition and kept pluripotency of ESCs by concentrating LIF at mechanically-agitated state. **P <0.01 (ANOVA).

FIG. 34 is an illustration of coacervate-mediated spatial macromolecular heterogeneity used to induce differential polarizations of encapsulated macrophage in the same pool of basal medium: coacervates labeled with alphabetic patterns “U” and “K” were pre-loaded with M1 and M2 inductive factors, respectively, while “C” and “H” coacervates were not loaded with any inductive factors.

FIG. 35 presents immunofluorescence staining images revealing differential expression of M1(iNOS)/M2(Arg-1) markers in the macrophages encapsulated in different coacervate alphabetic patterns. Scale bar: 20 μm.

FIG. 36 is a graph of qPCR results revealing differential expression of M2(Arg-1) and M1(iNOS) markers in the macrophages encapsulated in different coacervate alphabetic patterns. ***P <0.001 (ANOVA).

FIG. 37 illustrates coacervate-hydrogel transition via addition of Ti⁴⁺: vacuolated NPA coacervate was transformed into a self-healing NPA/Ti hydrogel stabilized by Ti⁴⁺—catechol coordination at pH 7.4.

FIG. 38 is a graph of frequency-dependent storage (G′) and loss (G″) moduli of the NPA/Ti hydrogel.

FIG. 39 is a graph of results from a shear-thinning test confirming the excellent self-healing ability of the NPA/Ti hydrogel.

FIG. 40 is a graph demonstrating that the NPA/Ti hydrogel is non-swellable due to the combination of the strong catechol-Ti⁴⁺ coordination bond and hydrophobic alkyl core of the assembled nanoparticle.

FIG. 41 presents images demonstrating that the coacervate-hydrogel transition disrupted vacuolated morphologies to generate the homogenous NPA/Ti hydrogel, as evidenced by the uniform distribution of both FITC and Texas Red-labeled BSA. Scale bar: 20 μm.

FIG. 42 presents fluorescence images (IVIS) showing that after oral gavage, NPA coacervates (modified with Cy7 tag) stay much longer than NPA-Phenyl coacervate at the GI tract.

FIG. 43 is a graph demonstrating that the provided NPA coacervate exhibits prolonged release of pre-loaded Dex-P in vitro. In contrast, due to the highly permeable structure, conventional PEG hydrogels with a similar solid content as that of the NPA coacervate released almost 80% of the pre-loaded Dex-P after 4 hours.

FIG. 44 is a graph showing the concentration of Dex in rat plasma after oral gavage. Dex-P/NPA coacervate group demonstrated lower burst increase of plasma Dex concentration than the Free Dex-P group.

FIG. 45 is a graph showing that, compared with the highly permeable PEG hydrogels with similar solid content, the condensed hydrophobic environment of NPA coacervate facilitated the sustained release of a wide array of water-soluble small-molecular drugs.

FIG. 46 illustrates the provided non-complex NPA coacervate compared with the conventional pH- and salt-dependent complex coacervate stabilized by electrostatic interactions between polyanions and polycations.

FIG. 47 illustrates that, driven by the gastrointestinal peristalsis, fluid NPA coacervate can effectively spread to coat and adhere on the large intestinal surface area via catechol-mediated wet bioadhesion.

FIG. 48 presents photographs showing the liquid-like (G′<G″) NPA coacervate (stained by Fast Green FCF) can be injected through a 21G needle and can remain stable in buffers with a wide range of pH after 2 days.

FIG. 49 is a graph showing that non-complex NPA coacervate exhibits a salting-out effect, confirming that the formation of NPA coacervates should be attributed to hydrogen bonding-induced nanoparticle assembly rather than electrostatic interactions.

FIG. 50 presents a photograph and illustrations showing that NPA coacervate can glue two ribbons of pork skin tissue together and hold the weight of the tissues.

FIG. 51 presents a series of photographs showing that the fluid NPA coacervate coating can adhere to fresh and wet mucosa, flow down slowly. Scale bar: 15 mm.

FIG. 52 presents photographs showing that the fluid NPA coacervate coating can remain stable after soaking in simulated gastric fluid (Ga) and simulated intestinal fluid (In) at 37° C. for 2 hours, respectively.

FIG. 53 illustrates the general procedure of an experiment using a rat model of DSS-induced colitis, in which SD rats were given 4.5% DSS in drinking water to induce acute colitis.

FIG. 54 illustrates the time course of the experiment of FIG. 53, in which the colitic rats received oral gavages of Dex-P-laden NPA coacervates (Dex-P/NPA) or the equivalent amount of Dex-P in PBS (Dex-P/PBS) on days 1, 3, and 5. Untreated colitic SD rats were used as the negative control (Control). All SD rats were sacrificed on day 7.

FIG. 55 present photographic results from the experiment of FIGS. 53 and 54, showing that colonic edema and diarrhea caused by DSS-induced colitis in SD rats receiving Dex-P/NPA were significantly relieved compared with that of the untreated colitic SD rats (Control) and colitic SD rats receiving Dex-P in PBS (Dex-P/PBS). Scale bar: 10 mm.

FIG. 56 is a graph of results from the experiment of FIGS. 53-55, further showing that colonic edema and diarrhea caused by DSS-induced colitis in SD rats receiving Dex-P/NPA were significantly relieved compared with that of the untreated colitic SD rats (Control) and colitic SD rats receiving Dex-P in PBS (Dex-P/PBS).

FIG. 57 presents representative images of H&E staining demonstrating that histological inflammation was diminished in the colitic SD rats from the experiment of FIGS. 53-56 receiving Dex-P/NPA, while histological damages were observed in untreated colitic SD rats (Control) or colitic SD rats receiving Dex-P/PBS. Scale bar: 150 μm.

FIG. 58 is a graph of MPO activity from the experiment of FIGS. 53-57. Data are presented as mean ±SD. *P<0.05, **P<0.01, ***P<0.001 (ANOVA).

FIG. 59 is a graph of mRNA levels of tight junction-associated proteins, including ZO-1 and occludin-1, from the experiment of FIGS. 53-58. Data are presented as mean±SD. *P<0.05, **P<0.01, ***P<0.001 (ANOVA).

FIG. 60 is a graph showing that Dex-P/NPA treatment increased bacterial richness in fecal samples collected on day 5 from randomly selected colitic SD rats and analyzed for gut microbiota by sequencing the V4 region of the 16S rRNA gene.

FIG. 61 presents graphs comparing Chao diversity and Shannon diversity in colitic SD rats with that in colitic SD rats in the Dex-P/PBS group and in the untreated colitic rats (Control).

FIG. 62 presents a clustered heatmap of gut microbiota β-diversity illustrating that colitic SD rats receiving Dex-P/NPA and healthy SD rats are clustered more closely, suggesting more similar bacterial compositions.

FIG. 63 presents a taxonomic bacterial distribution based on the relative abundance of the gut microbiota at family-level.

FIG. 64 presents a clustered heatmap based on the relative abundance of the gut microbiota of FIG. 63 at family-level. The upper longitudinal clustering indicates the similarity of gut microbiota among individual SD rats. The closer distance and shorter branch length indicate more similar gut microbiota between the SD rats. Data are presented as mean±SD. *P<0.05, **P<0.01, ***P<0.001 (ANOVA).

FIG. 65 is an illustration of formation of core-shell nanoparticles displaying surface catechol groups.

FIG. 66 illustrates mechanically triggered brief burst release of macromolecules segregated within coacervate vacuoles.

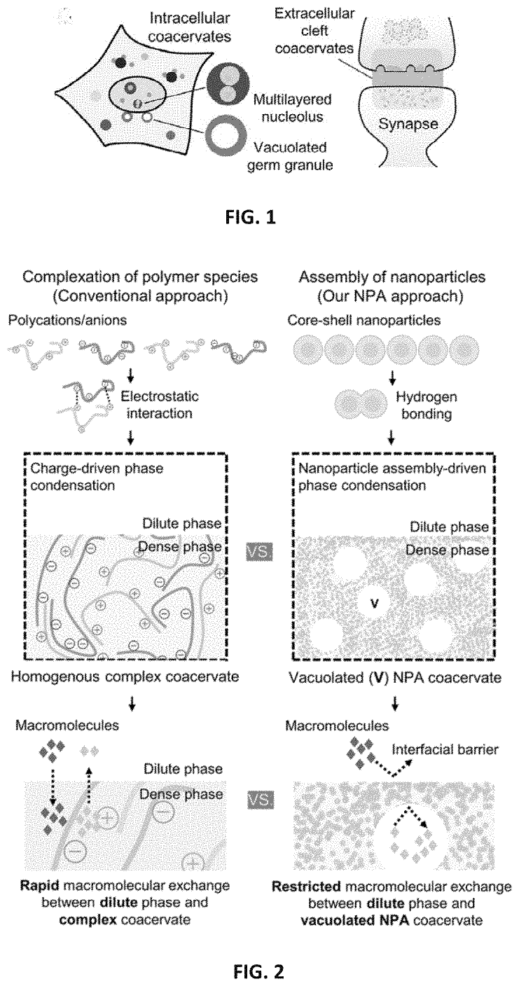

### DETAILED DESCRIPTION

### Coacervates

In one aspect of the present invention, a population of coacervates is disclosed. As used herein, the terms “coacervates” or “coacervate” refer to an organic-rich polymer-concentrated and water-immiscible liquid phase formed via liquid-liquid phase separation, and resulting from association of, e.g., molecules having different hydrophobicity and hydrophilicity, or having opposite ionic charges. Coacervate can thus exist as a denser matrix, layer, or droplet within a larger and more dilute liquid phase. This dense coacervate matrix can be, for example, a hydrogel. Additionally, the term “coacervate compartments” refers to further liquid-liquid subdivisions within the dense matrix creating compartments interior to the coacervate and having different, e.g., more liquid, properties. This compartment formation can therefore result in structures referred to as “vacuolated coacervate,” “coacervate microdroplets” or the like. The coacervates of the provided population each include an assembly of nanoparticles. As used herein, the term “nanoparticle” refers to any particle with a size that is in the range of nanometers. For example, a nanoparticle can have a diameter of less than 1 micron (1000 nm), or greater than 1 nm.

The nanoparticles of the coacervates (e.g., at least a portion of the nanoparticles, a majority of the nanoparticles, or all of the nanoparticles) each include a hydrophobic core and a plurality of hydrophilic polymeric chains. As used herein, the terms “polymeric” and “polymer” refer to an organic substance composed of a plurality of repeating structural units (monomeric units) covalently linked to one another.

In some embodiments, the nanoparticles each include an amphiphilic polymer, wherein the hydrophobic core of each nanoparticle includes hydrophobic segments of the amphiphilic polymer, and the hydrophilic polymeric chains of each nanoparticle include hydrophilic segments of the amphiphilic polymer. In certain embodiments, the hydrophobic segments of the amphiphilic polymer include alkyl groups, e.g., long-chain alkyl groups. As used herein, the term “alkyl” refers to straight or branched, saturated, aliphatic groups. In certain embodiments, the hydrophobic segments of the amphiphilic polymer include acrylamide groups. The hydrophobic segments can include, for example, poly(N-isopropylacrylamide). In some embodiments, the nanoparticles do not include an amphiphilic polymer, and the hydrophobic core of each nanoparticle includes hydrophobic polymers that can include, for example, alkyl and/or acrylamide segments.

In some embodiments, the hydrophobic core of each nanoparticle includes an inorganic material. In certain embodiments, the inorganic material includes gold-containing nanoparticles. In certain embodiments, the inorganic material includes iron-containing nanoparticles, e.g., iron oxide nanoparticles such as magnetite nanoparticles or maghemite nanoparticles. In certain embodiments, the inorganic material includes silica-containing nanoparticles. The hydrophobic core can include one type of inorganic material. The hydrophobic core can include two or more inorganic materials. The hydrophobic core can consist of one, two, three, four, five, six, seven, eight, nine, ten, or more than ten inorganic materials.

The hydrophilic polymeric chains of the nanoparticles form a hydrophilic shell about the hydrophobic core of each nanoparticle of the coacervates. In some embodiments, the hydrophilic polymeric chains of each nanoparticle include a polyether. In certain embodiments, the polyether include polyethylene glycol.

The hydrophilic polymeric chains (e.g., at least a portion of the hydrophilic polymeric chains, a majority of the hydrophilic polymeric chains, or all of the hydrophilic polymeric chains) each include a functional end group. The functional end groups are thus displayed on the surface of the hydrophilic shell of the nanoparticles of each coacervate. The functional end groups can be selected to provide non-covalent interactions to drive the assembly of the core-shell nanoparticles in the formation of the coacervates.

In some embodiments, the functional end group includes an aryl group. As used herein, the term “aryl” refers to an aromatic ring system having any suitable number of ring atoms and any suitable number of rings. Aryl groups can include any suitable number of ring atoms, such as 6, 7, 8, 9, 10, 11, 12, 13, 14, 15, or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl, and biphenyl. Other aryl groups include benzyl, having a methylene linking group. Some aryl groups have from 6 to 12 ring members, such as phenyl, naphthyl, or biphenyl. Other aryl groups have from 6 to 10 ring members, such as phenyl or naphthyl. Some other aryl groups have 6 ring members, such as phenyl. Aryl groups can be substituted or unsubstituted.

The aryl groups can optionally be substituted by any suitable number and type of subsituents. Representative substituents include, but are not limited to, halogen, haloalkyl, haloalkoxy, —OR′, ═O, —OC(O)R′, —(O)R′, —O₂R′, —ONR′R″, —OC(O)NR′R″, ═NR′, ═N—OR′, —NR′R″, —NR″C(O)R′, —NR′—(O)NR″R′″, —NR″C(O)OR′, —NH—(NH₂)═NH, —NR′C(NH₂)═NH, —NH—(NH₂)═NR′, —SR′, —S(O)R′, —S(O)₂R′, —S(O)₂NR′R″, —NR′S(O)₂R″, —N₃ and —NO₂. R′, R″ and R′″ each independently refer to hydrogen or unsubstituted alkyl, such as unsubstituted C₁₋₆ alkyl. Alternatively, R′ and R″, or R″ and R′″, when attached to the same nitrogen, are combined with the nitrogen to which they are attached to form a heterocycloalkyl or heteroaryl ring.

In certain embodiments, the functional end group includes phenyl. In some embodiments, the functional end group includes a hydroxylated aryl group. In certain embodiments, the hydroxylated aryl group includes a dihydroxybenzene. The dihydroxybenzene can include, for example, catechol. In some embodiments, the hydrophilic polymeric chains include catechol-grafted polyethylene glycol.

The coacervates further include non-covalent interactions between at least a portion of the functional end groups. The non-covalent interactions can be responsible for forming assemblies of the nanoparticles disclosed herein into coacervates. In certain embodiments, the non-covalent interactions include hydrogen bonds. In certain embodiments, the non-covalent interactions include π-π interactions. In certain embodiments, the non-covalent interactions include electrostatic interactions. In certain embodiments, the non-covalent interactions include cation-π interactions. The non-covalent interactions between at least a portion of the functional end groups can include one type of non-covalent interaction or multiple types of non-covalent interactions. In some embodiments, the non-covalent interactions include π-π interactions between phenyl end groups of the hydrophilic polymeric chains. In some embodiments, the non-covalent interactions include hydrogen bonds between catechol end groups of the hydrophilic polymeric chains.

The average hydrodynamic radius of the nanoparticles of the coacervates can be, for example, between 1 nm and 900 nm, e.g., between 1 nm and 59 nm, between 2 nm and 117 nm, between 4 nm and 231 nm, between 8 nm and 456 nm, or between 15 nm and 900 nm. In some embodiments, the average nanoparticle radius is between 30 nm and 300 nm, e.g., between 30 nm and 192 nm, between 57 nm and 219 nm, between 84 nm and 246 nm, between 111 nm and 273 nm, or between 138 nm and 300 nm. In terms of upper limits, the average nanoparticle radius can be less than 900 nm, e.g., less than 456 nm, less than 273 nm, less than 246 nm, less than 219 nm, less than 192 nm, less than 165 nm, less than 138 nm, less than 111 nm, less than 84 nm, less than 57 nm, less than 30 nm, less than 15 nm, less than 8 nm, less than 4 nm, or less than 2 nm. In terms of lower limits, the average nanoparticle radius can be greater than 1 nm, e.g., greater than 2 nm, greater than 4 nm, greater than 8 nm, greater than 15 nm, greater than 30 nm, greater than 57 nm, greater than 84 nm, greater than 111 nm, greater than 138 nm, greater than 165 nm, greater than 192 nm, greater than 219 nm, greater than 246 nm, greater than 273 nm, or greater than 456 nm. Larger radii, e.g., greater than 900 nm, and smaller radii, e.g., less than 1 nm, are also contemplated.

In some embodiments, the coacervates reversibly transition between a compartmentalized, e.g., vacuolated liquid or microdroplet, state and a hydrogel state as the temperature of the coacervates passes beyond an upper critical solution temperature. The coacervates can transition from a vacuolated liquid state to a hydrogel state as the temperature is decreased beyond the upper critical solution temperature. The coacervates can transition from a hydrogel state to a vacuolated liquid state as the temperature is increased beyond the upper critical solution temperature. The upper critical solution temperature can be, for example, between 2° C. and 40° C., e.g., between 2° C. and 24.8° C., between 5.8° C. and 28.6° C., between 9.6° C. and 32.4° C., between 13.4° C. and 36.2° C., or between 17.2° C. and 40° C. The upper critical solution temperature can be, for example, between 2° C. and 20° C., e.g., between 2° C. and 12.8° C., between 3.8° C. and 14.6° C., between 5.6° C. and 16.4° C., between 7.4° C. and 18.2° C., or between 9.2° C. and 20° C. In terms of upper limits, the upper solution critical temperature can be less than 40° C., e.g., less than 36.2° C., less than 32.4° C., less than 28.6° C., less than 24.8° C., less than 21° C., less than 18.2° C., less than 16.4° C., less than 14.6° C., less than 12.8° C., less than 11° C., less than 9.2° C., less than 7.4° C., less than 5.6° C., or less than 3.8° C. In terms of lower limits, the upper solution critical temperature can be greater than 2° C., e.g., greater than 3.8° C., greater than 5.6° C., greater than 7.4° C., greater than 9.2° C., greater than 11° C., greater than 12.8° C., greater than 14.6° C., greater than 16.4° C., greater than 18.2° C., greater than 21° C., greater than 24.8° C., greater than 28.6° C., greater than 32.4° C., or greater than 36.2° C. Higher temperatures, e.g., greater than 40° C., and lower temperatures, e.g., less than 2° C., are also contemplated.

As discussed above, the provided coacervates and coacervate compartments can demonstrate the advantageous property of increased resistance to coalescence under common physiological conditions. Evidence of this coalescence resistance can be seen in coacervate or vacuole dimensions that do not expand too significantly under such conditions. The average diameter of the provided vacuoles within coacervates upon storage for 6 hours at 37° C. and pH 7.4 can be, for example, between 10 μm and 100 μm, e.g., between 10 μm and 64 μm, between 19 μm and 73 μm, between 28 μm and 82 μm, between 37 μm and 91 μm, or between 46 μm and 100 μm. In terms of upper limits, the average coacervate diameter can be less than 100 μm, e.g., less than 91 μm, less than 82 μm, less than 73 μm, less than 64 μm, less than 55 μm, less than 46 μm, less than 37 μm, less than 28 μm, or less than 19 μm. In terms of lower limits, the average coacervate diameter can be greater than 10 μm, e.g., greater than 19 μm, greater than 28 μm, greater than 37 μm, greater than 46 μm, greater than 55 μm, greater than 64 μm, greater than 73 μm, greater than 82 μm, or greater than 91 μm. Larger diameters, e.g., greater than 100 μm, and smaller diameters, e.g., less than 10 μm, are also contemplated.

A related advantageous property of the provided coacervates and coacervate compartments is their relatively small degree of polydispersity, e.g., upon storage in physiological conditions that promote the coalescence of conventionally prepared coacervates. The standard deviation of the diameters of the provided coacervates upon storage for 6 hours at 37° C. and pH 7.4 can be, for example, between 4% and 40%, e.g., between 4% and 25.6%, between 7.6% and 29.2%, between 11.2% and 32.8%, between 14.8% and 36.4%, or between 18.4% and 40%, In terms of upper limits, the coacervate diameter standard deviation can be less than 40%, e.g., less than 36.4%, less than 32.8%, less than 29.2%, less than 25.6%, less than 22%, less than 18.4%, less than 14.8%, less than 11.2%, or less than 7.6%. In terms of lower limits, the coacervate diameter standard deviation can be greater than 4%, e.g., greater than 7.6%, greater than 11.2%, greater than 14.8%, greater than 18.4%, greater than 22%, greater than 25.6%, greater than 29.2%, greater than 32.8%, or greater than 36.4%. Larger standard deviations, e.g., greater than 40%, and smaller standard deviations, e.g., less than 4%, are also contemplated.

A beneficial attribute of the provided coacervates is their ability to serve as microcompartments suitable for creating and/or preserving spatiotemporal heterogeneity of materials such as macromolecules. As used herein, the term “macromolecule” refers to a molecule of high relative molecular mass, the structure of which can comprises the multiple repetition of units derived from molecules of low relative molecular mass, e.g., monomers and/or oligomers. The macromolecule can be, for example and without limitation, a protein, a nucleic acid, a carbohydrate, a lipid, a macrocycle, or a synthetic polymer The microcompartment ability of the coacervates is dependent on the coacervate matrix having a sufficiently high barrier efficiency to minimize or prevent exchange of material between the vacuoles and the medium exterior to the coacervates. As used herein, the term “barrier efficiency” refers to the ratio of the concentration of a molecule in the medium outside of the coacervate to the concentration of that molecule within the coacervate, e.g., within the vacuoles of a vacuolated coacervate. The barrier efficiency can readily be measured through, for example, fluorescence intensity measurements of a fluorescent or fluorescently tagged molecule, such that the barrier efficiency can be calculated from the mean fluorescence intensity of the molecule in the volumes exterior and interior to the coacervates or vacuoles.

The barrier efficiency of the coacervates with respect to fluorescein isothiocyanate conjugated bovine serum albumin (FITC-BSA) upon storage for 1 day in a buffer including FITC-BSA can be, for example, between 5 and 50, e.g., between 5 and 32, between 9.5 and 36.5, between 14 and 41, between 18.5 and 45.5, or between 23 and 50. In terms of upper limits, the coacervate barrier efficiency with respect to FITC-BSA can be less than 50, e.g., less than 45.5, less than 41, less than 36.5, less than 32, less than 27.5, less than 23, less than 18.5, less than 14, or less than 9.5. In terms of lower limits, the coacervate barrier efficiency with respect to FITC-BSA can be greater than 5, e.g., greater than 9.5, greater than 14, greater than 18.5, greater than 23, greater than 27.5, greater than 32, greater than 36.5, greater than 41, or greater than 45.5. Higher barrier efficiencies, e.g., greater than 50, and lower barrier efficiencies, e.g., less than 5, are also contemplated.

The barrier efficiency of the coacervates with respect to Texas Red conjugated bovine serum albumin (Texas Red-BSA) upon storage for 1 day in a buffer including Texas Red-BSA can be, for example, between 5 and 50, e.g., between 5 and 32, between 9.5 and 36.5, between 14 and 41, between 18.5 and 45.5, or between 23 and 50. In terms of upper limits, the coacervate barrier efficiency with respect to Texas Red-BSA can be less than 50, e.g., less than 45.5, less than 41, less than 36.5, less than 32, less than 27.5, less than 23, less than 18.5, less than 14, or less than 9.5. In terms of lower limits, the coacervate barrier efficiency with respect to Texas Red-BSA can be greater than 5, e.g., greater than 9.5, greater than 14, greater than 18.5, greater than 23, greater than 27.5, greater than 32, greater than 36.5, greater than 41, or greater than 45.5. Higher barrier efficiencies, e.g., greater than 50, and lower barrier efficiencies, e.g., less than 5, are also contemplated.

In some instances and applications, it is beneficial that the provided coacervates to have a sufficiently high uptake efficiency to permit or promote exchange of material between the volumes interior and exterior to the coacervates. As used herein, the term “uptake efficiency” refers to the ratio of the concentration of a molecule within the coacervate, e.g., within the vacuoles of a vacuolated coacervate, to the concentration of that molecule in the medium outside of the coacervate. The uptake efficiency can readily be measured through, for example, fluorescence intensity measurements of a fluorescent or fluorescently tagged molecule, such that the uptake efficiency can be calculated from the mean fluorescence intensity of the molecule in the volumes exterior and interior to the coacervates. The uptake efficiency can also or alternatively be measured by observing absorbance of ultraviolet or visible light associated with a molecule, such that the uptake efficiency can be calculated from the mean absorbance value, e.g., at a specific wavelength, of the molecule in the volumes exterior and interior to the coacervate.

In some embodiments, the provided coacervates demonstrate the advantageous characteristic of having an uptake efficiency that can be activated, i.e., increased, upon a perturbation, e.g., mechanical agitation. The uptake efficiency of the coacervates with respect to FITC-BSA upon agitation for 10 seconds at 3000 RPM can be, for example, between 0.5 and 5, e.g., between 0.5 and 3.2, between 0.95 and 3.65, between 1.4 and 4.1, between 1.85 and 4.55, or between 2.3 and 5. In terms of upper limits, the coacervate uptake efficiency with respect to FITC-BSA can be less than 5, e.g., less than 4.55, less than 4.1, less than 3.65, less than 3.2, less than 2.75, less than 2.3, less than 1.85, less than 1.4, or less than 0.95. In terms of lower limits, the coacervate barrier efficiency with respect to FITC-BSA can be greater than 0.5, e.g., greater than 0.95, greater than 1.4, greater than 1.85, greater than 2.3, greater than 2.75, greater than 3.2, greater than 3.65, greater than 4.1, or greater than 4.55. Higher uptake efficiencies, e.g., greater than 5, and lower barrier efficiencies, e.g., less than 0.5, are also contemplated.

The uptake efficiency of the coacervate with respect to BSA upon agitation for 10 seconds at 3000 RPM can be, for example, between 5% and 50%, e.g., between 5% and 32%, between 9.5% and 36.5%, between 14% and 41%, between 18.5% and 45.5%, or between 23% and 50%. In terms of upper limits, the coacervate uptake efficiency can be less than 50%, e.g., less than 45.5%, less than 41%, less than 36.5%, less than 32%, less than 27.5%, less than 23%, less than 18.5%, less than 14%, or less than 9.5%. In terms of lower limits, the coacervate uptake efficiency can be greater than 5%, e.g., greater than 9.5%, greater than 14%, greater than 18.5%, greater than 23%, greater than 27.5%, greater than 32%, greater than 36.5%, greater than 41%, or greater than 45.5%. Higher uptake efficiencies, e.g., greater than 50%, and lower uptake efficiencies, e.g., less than 5%, are also contemplated.

### Methods of Forming Coacervates

Another aspect of the present invention is a method of forming a population of coacervates. The method includes providing a population of polymers. The provided polymers can be any of those disclosed herein and including hydrophilic polymeric chains, each of which includes a functional end group.

The method further includes fabricating, via self-assembly of the polymers, a population of nanoparticles. The fabricated nanoparticles can be any of those disclosed herein and including a hydrophobic core and a plurality of hydrophilic polymeric chains extending from the hydrophobic core. In some embodiments, each polymer is an amphiphilic polymer, wherein the hydrophobic core of the nanoparticles include hydrophobic segments of the amphiphilic polymer, and the hydrophilic polymeric chains of the nanoparticles include hydrophilic segments of the amphiphilic polymer. The hydrophobic segments of the amphiphilic polymer can be any of those disclosed herein. In certain embodiments, the hydrophobic segments include alkyl groups. In some embodiments, the hydrophobic core of the nanoparticles includes an inorganic material. The inorganic material can be any of those disclosed herein. In some embodiments, the hydrophilic polymeric chains of the nanoparticles include a polyether. In certain embodiments, the polyether includes a polyethylene glycol. In some embodiments, the functional end group of hydrophilic polymeric chains includes a hydroxylated aryl group. In certain embodiments, the hydroxylated aryl group includes a dihydroxybenzene. The dihydroxybenzene can include, for example, catechol. In some embodiments, the hydrophilic polymeric chains include catechol-grafted polyethylene glycol.

The method further includes forming, via non-covalent interactions between at least a portion of the functional end groups, the population of coacervates. The non-covalent interactions can be any of those disclosed herein. In some embodiments, the forming includes dialyzing a suspension of the population of nanoparticles against water. The dialyzing can be performed at a temperature, for example, between 15° C. and 30° C., e.g., between 15° C. and 24° C., between 16.5° C. and 25.5° C., between 18° C. and 27° C., between 19.5° C. and 28.5° C., or between 21° C. and 30° C. In terms of upper limits, the dialysis temperature can be less than 30° C., e.g., less than 28.5° C., less than 27° C., less than 25.5° C., less than 24° C., less than 22.5° C., less than 21° C., less than 19.5° C., less than 18° C., or less than 16.5° C. In terms of lower limits, the dialysis temperature can be greater than 15° C., e.g., greater than 16.5° C., greater than 18° C., greater than 19.5° C., greater than 21° C., greater than 22.5° C., greater than 24° C., greater than 25.5° C., greater than 27° C., or greater than 28.5° C. Higher temperatures, e.g., greater than 30° C., and lower temperatures, e.g., less than 15° C., are also contemplated.

The dialyzing can be performed for a time, for example, between 24 hours and 72 hours, e.g., between 24 hours and 52.8 hours, e.g., between 28.8 hours and 57.6 hours, between 33.6 hours and 62.4 hours, between 38.4 hours and 67.2 hours, or between 43.2 hours and 72 hours. In terms of upper limits, the dialysis time can be less than 72 hours, e.g., less than 67.2 hours, less than 62.4 hours, less than 57.6 hours, less than 52.8 hours, less than 48 hours, less than 43.2 hours, less than 38.4 hours, less than 33.6 hours, or less than 28.8 hours. In terms of lower limits, the dialysis time can be greater than 24 hours, e.g., greater than 28.8 hours, greater than 33.6 hours, greater than 38.4 hours, greater than 43.2 hours, greater than 48 hours, greater than 52.8 hours, greater than 57.6 hours, greater than 62.4 hours, or greater than 67.2 hours. Longer dialysis times, e.g., greater than 72 hours, and shorter dialysis times, e.g., less than 24 hours, are also contemplated.

Methods of Controlling Macromolecular Distribution using Coacervates

As discussed above, a beneficial attribute of the provided coacervates, e.g., vacuolated coacervates, is their ability to serve as microcompartments suitable for creating and/or preserving spatiotemporal heterogeneity of materials such as macromolecules. In some embodiments, the vacuolated coacervates are useful for creating such heterogeneity by encapsulating one or more types of materials. In certain embodiments, the provided vacuolated coacervates encapsulate a bioactive compound or a population of cells. As used herein, the term “bioactive” refers to a compound having a physiological effect on a biological system or subject as compared to a biological system or subject not exposed to the compound. The encapsulated population of cells can include primarily or entirely one species or strain of cells. The encapsulated population of cells can include multiple species or strains of cells.

The ability of the provided vacuolated coacervates to compartmentalize materials relies on barriers to transport that are created when the coacervates are in a vacuolated physiological state having clearly defined interior and exterior volumes. In contrast, this transport barrier is fully or partially removed if the coacervates lose their vacuolated form and transition to a homogeneous hydrogel physiological state. Such a hydrogel state, while being less effective at establishing and maintaining localized concentrations of materials, can be useful in creating a more homogeneous matrix capable of, e.g., supporting the growth and structure of cellular colonies. The nanoparticle-assembled coacervates disclosed herein possess the advantageous trait of being able to transition from one of these two physiological states to another in the presence of various external stimuli, e.g., contact with metal ions, changes in temperature, and/or mechanical agitation.

Accordingly, another aspect of the present invention is a method of transitioning a population of coacervates from a vacuolated state to a hydrogel state through contacting the coacervates with metal ions. The method can include, for example, contacting the vacuolated coacervate with titanium ions, e.g., Ti⁴⁺, thereby transitioning the coacervate to a hydrogel state. The method can include contacting the vacuolated coacervate with iron ions, e.g., Fe³⁺. The method can include contacting the vacuolated coacervate with aluminum ions, e.g., Al³⁺.

The method can include contacting the vacuolated coacervate with nickel ions, e.g., Ni²⁺. The method can include contacting the vacuolated coacervate with copper ions, e.g., Cu²⁺. The method can include contacting the vacuolated coacervate with zinc ions, e.g., Zn²⁺. The method can include contacting the vacuolated coacervate with one species of metal ion. The method can include contacting the vacuolated coacervate with two or more species of metal ions, either sequentially or simultaneously.

Another aspect of the present invention is a method of reversibly switching the physiological state of a population of coacervates. In certain embodiments, the method involves reversibly switching the coacervate physical state from a vacuolated liquid state to a hydrogel state and vice versa through changing the temperature of the coacervates as disclosed herein. In particular, the method includes passing the temperature of the coacervates beyond an upper critical solution temperature.

Another aspect of the present invention is a method of transiently activating uptake of a macromolecule by a population of coacervates. This uptake activation is achieved through a temporary change of the coacervate physiological state from a vacuolated liquid state having a relatively high barrier efficiency and low uptake efficiency to a hydrogel state having a relative high uptake efficiency and low barrier efficiency. By using such a method, the coacervates can, for example, be loaded with material when the coacervate uptake efficiency is higher, and effectively compartmentalize the material when the coacervate returns to having a higher barrier efficiency. The method includes providing a population of coacervates as disclosed herein. The method further includes agitating the coacervates in a solution, e.g., a buffer, including the macromolecule. The agitating increases the uptake efficiency of the vacuolated coacervates, and activates uptake of the macromolecule by the vacuolated coacervates. The agitating can be performed, for example, for 10 seconds at 3000 RPM. The method further include stopping the agitation of the vacuolated coacervates, thereby increasing the barrier efficiency of the vacuolated coacervates.

For example, a layered NPA coacervate matrix, e.g., a collection of assembled nanoparticles with dense hydrophobic cores and PEG chains, can act as a highly molecularly crowded barrier to isolate the internal vacuoles from surroundings under a close to an equilibrium condition. An applied mechanical agitation can then transiently disrupt the NPA coacervate matrix and create a window of opportunity for macromolecules to be enclosed into the vacuoles.

The uptake efficiency increase of the coacervates with respect to the macromolecule upon the agitating can be, for example, between 10-fold and 100-fold, e.g., between 10-fold and 64-fold, between 19-fold and 73-fold, between 28-fold and 82-fold, between 37-fold and 91-fold, or between 46-fold and 100-fold. In terms of upper limits, the uptake efficiency increase can be less than 100-fold, e.g., less than 91-fold, less than 82-fold, less than 73-fold, less than 64-fold, less than 55-fold, less than 46-fold, less than 37-fold, less than 28-fold, or less than 19-fold. In terms of lower limits, the uptake efficiency increase can be greater than 10-fold, e.g., greater than 19-fold, greater than 28-fold, greater than 37-fold, greater than 46-fold, greater than 55-fold, greater than 64-fold, greater than 73-fold, greater than 82-fold, or greater than 91-fold. Larger increases, e.g., greater than 100-fold, and smaller increases, e.g., less than 10-fold, are also contemplated.

The uptake efficiency increase of the coacervate with respect to the macromolecule upon the agitating can be, for example, between 5% and 50%, e.g., between 5% and 32%, between 9.5% and 36.5%, between 14% and 41%, between 18.5% and 45.5%, or between 23% and 50%. In terms of upper limits, the uptake efficiency increase can be less than 50%, e.g., less than 45.5%, less than 41%, less than 36.5%, less than 32%, less than 27.5%, less than 23%, less than 18.5%, less than 14%, or less than 9.5%. In terms of lower limits, the uptake efficiency increase can be greater than 5%, e.g., greater than 9.5%, greater than 14%, greater than 18.5%, greater than 23%, greater than 27.5%, greater than 32%, greater than 36.5%, greater than 41%, or greater than 45.5%. Larger increases, e.g., greater than 50%, and smaller increases, e.g., less than 5%, are also contemplated.

### Methods of Delivering Coacervates

The improved stability and polydispersity of the provided nanoparticle-assembled coacervates under physiological conditions allow the coacervates to be particularly useful in methods involving the delivery of coacervates by administering to a subject. The delivery could include the use of the coacervates, e.g., vacuolated coacervates, as delivery vehicles for one or more materials useful in a therapy or treatment, e.g., a therapeutic agent or a detectable agent. The delivery could include the use of the coacervates themselves as the active agent of a therapy or treatment. As used herein, the term “administering” refers to oral administration, administration as a suppository, topical contact, parenteral, intravenous, intraperitoneal, intramuscular, intralesional, intranasal or subcutaneous administration, or intrathecal administration to the subject. The coacervates of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The provided coacervates can be administered to a subject in any suitable amount, dependent on various factors including, but not limited to, the weight and age of the subject. Suitable dosage ranges for the coacervates of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The provided coacervates can be administered at any suitable frequency, interval and duration. For example, the coacervates can be administered once an hour, or two, three or more times an hour, once a day, or two, three, or more times per day, or once every 2, 3, 4, 5, 6, or 7 days, so as to provide the preferred dosage level. When the coacervates are administered more than once a day, representative intervals include 5, 10, 15, 20, 30, 45 and 60 minutes, as well as 1, 2, 4, 6, 8, 10, 12, 16, 20, and 24 hours. The coacervates can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The coacervates can also contain other compatible therapeutic agents. The provided coacervates can be used in combination with other active agents known to be useful in, e.g., obesity treatment, or with agents that may not be effective alone, but may contribute to the efficacy of the coacervates.

The provided coacervates can be co-administered with another active agent. Co-administration includes administering the coacervates and active agent within 0.5, 1, 2, 4, 6, 8, 10, 12, 16, 20, or 24 hours of each other. Co-administration also includes administering the coacervates and active agent simultaneously, approximately simultaneously (e.g., within about 1, 5, 10, 15, 20, or 30 minutes of each other), or sequentially in any order. Moreover, the coacervates and the active agent can each be administered once a day, or two, three, or more times per day so as to provide the preferred dosage level per day.

In some embodiments, co-administration can be accomplished by co-formulation, i.e., preparing a single pharmaceutical composition including both the coacervates and the active agent. In other embodiments, the coacervates and the active agent can be formulated separately.

The provided coacervates and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The coacervates and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the coacervates and the active agent are suitable in the compositions and methods disclosed herein.

In some embodiments, the targeted delivery of the coacervates to a subject is enhanced through bioadhesion of the coacervates to one or more surfaces within or on a subject body. As used herein, the term “subject” refers to animals such as mammals, including, but not limited to, primates (e.g., humans), cows, sheep, goats, horses, dogs, cats, rabbits, rats, mice and the like. In certain embodiments, the subject is a human. Bioadhesion of the coacervates can include a controlled macromolecular distribution of coacervates adhering to the subject body. Such functionality is difficult or impossible to achieve through the use of coacervates formed via conventional methods of complexation between oppositely-charged polymers.

In contrast, the use of selected chemistries, e.g., catechol groups, as the functional end groups of the hydrophilic polymeric chains of the nanoparticles of the coacervates provided herein allows for the formation of coacervates with good bioadhesion. This is because the selected end groups not only have non-covalent interactions with one another as required for nanoparticle assembly and coacervate formation, but also bond with biological surfaces to generate bioadhesion. In certain embodiments, the functional end group that selected to provide nanoparticle-assembled coacervate formation and bioadhesion includes catechol. In certain embodiments, the functional end group selected to provide nanoparticle-assembled coacervate formation and bioadhesion includes one or more nucleobases, e.g., adenine, thymine, cytosine, guanine, uracil, xanthine, hypoxanthine, purine, 2,6-diaminopurine, 6,8-diaminopurine, 5-methylcytosine, pseudouridine, dihydrouridine, inosine, 7-methylguanosine, xanthosine, 7-methylguanosine, dihydrouracil, hydroxymethylcytosine, methylcytidine, other modified or artificial nucleobases, or combinations thereof.

Accordingly, another aspect of the present invention is a method of adhering a population of coacervates within the body of a subject. The method includes providing a population of coacervates as disclosed herein, wherein the functional end group of the hydrophilic polymeric chains of the nanoparticles of the coacervates includes catechol. The method further includes administering the coacervates to the subject, thereby adhering the coacervates within the body of the subject. In certain embodiments, the administered coacervates adhere to the gastrointestinal tract of the subject. In certain embodiments, the administered coacervates encapsulate a bioactive compound. The bioactive compound can be an of those disclosed herein.

Another aspect of the present invention is a method of delivering a compound, e.g., a therapeutic compound, to a subject in need thereof. The method includes providing a population of coacervates as disclosed herein, wherein the coacervates encapsulate a therapeutically effective amount of the compound. In certain embodiments, the compound is a bioactive compound as disclosed herein. The method further includes administering the coacervates to the subject, thereby delivering the compound.

Another aspect of the present invention is a method of treating obesity. In some embodiments, the provided coacervates can adhere to the digestive tract of a subject, e.g., a subject suffering from obesity. Once thus adhered, the coacervates can reduce the uptake of nutrients by the subject through his or her digestive tract. Accordingly, the provided obesity treatment method includes administering to a subject in need thereof, a therapeutically effective amount of a population of coacervates as disclosed herein, wherein the functional end group of the hydrophilic polymeric chains of the nanoparticles of the coacervates includes catechol. Through the administering of the coacervates, at least a portion of the coacervates adhere to the gastrointestinal tract of the subject.

Another aspect of the present invention is a method of treating a digestive tract disorder, e.g., an inflammatory bowel disease (IBD) such as ulcerative colitis or Chron's disease. In some embodiments, the provided coacervates can adhere to the digestive tract of a subject, e.g., a subject suffering from an inflammatory bowel disease. In some embodiments, the provided coacervates can encapsulate one or more bioactive compounds, e.g., drugs, that are therapeutically effective in the treatment of an inflammatory bowel disease. The coacervates can thus adhere to the digestive tract of the subject, and deliver the one or more bioactive compounds, e.g., in a targeted fashion, to the digestive tract. Accordingly, a provided inflammatory bowel disease treatment method includes providing a population of coacervates as disclosed herein, wherein the coacervates encapsulate a therapeutically effective amount of a compound for treating an inflammatory bowel disease, and wherein the functional end group of the hydrophilic polymeric chains of the nanoparticles of the coacervates includes catechol. The method further includes administering the population of coacervates to a subject in need thereof. Through the administering of the coacervates, at least a portion of the coacervates adhere to the gastrointestinal tract of the subject, thereby delivering the compound.

### Compositions Including Coacervates

Another aspect of the present invention is a composition including a population of coacervates, e.g., vacuolated coacervates, as disclosed herein and a physiologically or pharmaceutically acceptable carrier, diluent, or excipient. As used herein, the term “composition” is intended to encompass a product comprising the specified ingredients in the specified amounts, as well as any product, which results, directly or indirectly, from combination of the specified ingredients in the specified amounts. By “physiologically/pharmaceutically acceptable” it is meant the carrier, diluent, or excipient must be compatible with the other ingredients of a formulation composition, suitable for the intended use of the composition so formulated, e.g., for administration to a live subject such as a human or animal, and not deleterious to the recipient thereof.

Pharmaceutically acceptable excipients are substance that aid the administration of the coacervates to and absorption by a subject. Pharmaceutical excipients suitable for use in the present disclosure include, but are not limited to, binders, fillers, disintegrants, lubricants, coatings, sweeteners, flavors and colors. One of skill in the art will recognize that other pharmaceutical excipients are useful in the present disclosure.

The compositions of the present disclosure can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, 351 J. Clin. Pharmacol. 1187 (1995); Tjwa, 75 Ann. Allergy Asthma Immunol. 107 (1995))).

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by combining the coacervates with suitable colorants, flavors, stabilizers, and thickening agents as desired. Aqueous suspensions suitable for oral use can be made by dispersing the coacervates in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

### Embodiments

The following embodiments are contemplated. All combinations of features and embodiment are contemplated.

Embodiment 1: A population of coacervates, each comprising an assembly of nanoparticles, wherein each nanoparticle comprises a hydrophobic core and a plurality of hydrophilic polymeric chains extending from the hydrophobic core, wherein the hydrophilic polymeric chains each comprise a functional end group, and wherein the coacervates further comprise non-covalent interactions between at least a portion of the functional end groups

Embodiment 2: An embodiment of embodiment 1, wherein the nanoparticles each comprise an amphiphilic polymer, wherein the hydrophobic core comprises hydrophobic segments of the amphiphilic polymer, and wherein the hydrophilic polymeric chains comprise hydrophilic segments of the amphiphilic polymer.

Embodiment 3: An embodiment of embodiment 2, wherein the hydrophobic segments comprise alkyl groups.

Embodiment 4: An embodiment of embodiment 1, wherein the hydrophobic core comprises an inorganic material.

Embodiment 5: An embodiment of any of the embodiments of embodiment 1-4, wherein the hydrophilic polymeric chains comprise a polyether.

Embodiment 6: An embodiment of embodiment 5, wherein the polyether comprises polyethylene glycol.

Embodiment 7: An embodiment of any of the embodiments of embodiment 1-6, wherein the functional end group comprises a hydroxylated aryl group.

Embodiment 8: An embodiment of embodiment 7, wherein the hydroxylated aryl group comprises a dihydroxybenzene.

Embodiment 9: An embodiment of embodiment 8, wherein the dihydroxybenzene comprises catechol.

Embodiment 10: An embodiment of any of the embodiments of embodiment 1-9, wherein the hydrophilic polymeric chains comprise catechol-grafted polyethylene glycol.

Embodiment 11: An embodiment of any of the embodiments of embodiment 1-10, wherein the nanoparticles have an average hydrodynamic radius between 1 nm and 900 nm.

Embodiment 12: An embodiment of any of the embodiments of embodiment 1-11, wherein the coacervates reversibly transition from a vacuolated liquid state to a hydrogel state as the temperature of the coacervates is decreased beyond an upper critical solution temperature.

Embodiment 13: An embodiment of embodiment 12, wherein the upper critical solution temperature is between 2° C. and 40° C.

Embodiment 14: An embodiment of any of the embodiments of embodiment 1-13, wherein vacuoles within the coacervates have an average diameter less than 100 μm upon storage for 6 hours at 37° C. and pH 7.4.

Embodiment 15: An embodiment of embodiment 14, wherein the standard deviation of the diameters of the vacuoles upon storage for 6 hours at 37° C. and pH 7.4 is less than 40%.

Embodiment 16: An embodiment of any of the embodiments of embodiment 1-15, wherein the barrier efficiency of the coacervates with respect to sulforhodamine 101 acid chloride conjugated bovine serum albumin (Texas Red-BSA) is greater than 5 upon storage for 1 day in a buffer comprising Texas Red-BSA.

Embodiment 17: An embodiment of embodiment 16, wherein the uptake efficiency of the coacervates with respect to BSA is greater than 5% upon agitation for 10 seconds at 3000 RPM in a buffer comprising BSA.

Embodiment 18: A method of forming a population of coacervates, the method comprising: providing a population of polymers, wherein each polymer comprises hydrophilic polymeric chains, and wherein each hydrophilic polymeric chain comprises a functional end group; fabricating, via self-assembly of the polymers, a population of nanoparticles, wherein each nanoparticle comprises a hydrophobic core, and wherein each nanoparticle further comprises a plurality of the hydrophilic polymeric chains extending from the hydrophobic core; and forming, via non-covalent interactions between at least a portion of the functional end groups, the population of coacervates.

Embodiment 19: An embodiment of embodiment 18, wherein each polymer is an amphiphilic polymer, wherein the hydrophobic core comprises hydrophobic segments of the amphiphilic polymer, and wherein the hydrophilic polymeric chains comprise hydrophilic segments of the amphiphilic polymer.

Embodiment 20: An embodiment of embodiment 19, wherein the hydrophobic segments comprise alkyl groups.

Embodiment 21: An embodiment of embodiment 18, wherein the hydrophobic core comprises an inorganic material.

Embodiment 22: An embodiment of any of the embodiments of embodiment 18-21, wherein the hydrophilic polymeric chains comprise a polyether.

Embodiment 23: An embodiment of embodiment 22, wherein the polyether comprises polyethylene glycol.

Embodiment 24: An embodiment of any of the embodiments of embodiment 18-23, wherein the functional end group comprises a hydroxylated aryl group.

Embodiment 25: An embodiment of embodiment 24, wherein the hydroxylated aryl group comprises a dihydroxybenzene.

Embodiment 26: An embodiment of embodiment 25, wherein the dihydroxybenzene comprises catechol.

Embodiment 27: An embodiment of any of the embodiments of embodiment 18-26, wherein the hydrophilic polymeric chains comprise catechol-grafted polyethylene glycol.

Embodiment 28: An embodiment of any of the embodiments of embodiment 18-27, wherein the forming comprises dialyzing a suspension of the population of nanoparticles against water.

Embodiment 29: An embodiment of embodiment 28, wherein the dialyzing is performed at a temperature between 15° C. and 30° C.

Embodiment 30: An embodiment of embodiment 28 or 29, wherein the dialyzing is performed for less than 3 days.

Embodiment 31: An embodiment of any of the embodiments of embodiment 18-30, wherein the population of nanoparticles has an average hydrodynamic radius between 1 nm and 900 nm.

Embodiment 32: A method of reversibly switching the physiological state of a population of coacervates, the method comprising: providing the population of coacervates of embodiment 1, wherein the coacervates have a first physiological state; and changing the temperature of the coacervates beyond an upper critical solution temperature, thereby switching the physiological state of the coacervates from the first physiological state to a second physiological state.

Embodiment 33: An embodiment of embodiment 32, further comprising: subsequent to the changing, adjusting the temperature of the coacervates beyond the upper critical solution temperature, thereby returning the physiological state of the coacervates from the second physiological state to the first physiological state.

Embodiment 34: An embodiment of embodiment 32 or 33, wherein the first physiological state is a vacuolated liquid state, wherein the changing comprises decreasing the temperature, and wherein the second physiological state is a hydrogel state.

Embodiment 35: An embodiment of embodiment 32 or 33, wherein the first physiological state is a hydrogel state, wherein the changing comprises increasing the temperature, and wherein the second physiological state is a vacuolated liquid state.

Embodiment 36: An embodiment of any of the embodiments of embodiment 32-35, wherein the upper critical solution temperature is between 2° C. and 40° C.

Embodiment 37: A method of transitioning a population of coacervates from a vacuolated liquid state to a hydrogel state, the method comprising: providing the population of coacervates of embodiment 1, wherein the coacervates have a vacuolated liquid state; and contacting the coacervates with Ti⁴⁺ titanium ions, thereby transitioning the coacervates to a hydrogel state.

Embodiment 38: An embodiment of embodiment 37, wherein the provided coacervates encapsulate a bioactive compound or a population of cells into vacuoles.

Embodiment 39: A method of transiently activating uptake of a macromolecule by a population of coacervates, the method comprising: providing the population of coacervates of embodiment 1; agitating the coacervates in a buffer comprising the macromolecule, thereby increasing the uptake efficiency of the coacervates and activating uptake of the macromolecule by the coacervates; and stopping the agitation of the coacervates, thereby increasing the barrier efficiency of the coacervates.

Embodiment 40: An embodiment of embodiment 39, wherein the uptake efficiency increase upon the agitating is greater than 5%.

Embodiment 41: An embodiment of embodiment 39 or 40, further comprising: encapsulating cells into the coacervates.

Embodiment 42: A method of adhering a population of coacervates within the body of a subject, the method comprising: providing the population of coacervates of embodiment 1, wherein the functional end group comprises catechol; and administering the coacervates to the subject, thereby adhering the coacervates within the body of the subject.

Embodiment 43: An embodiment of embodiment 42, wherein the coacervates adhere to the gastrointestinal tract of the subject.

Embodiment 44: An embodiment of embodiment 42 or 43, wherein the coacervates encapsulate a bioactive compound.

Embodiment 45: A method of delivering a compound to a subject in need thereof, the method comprising: providing the population of coacervates of any of the embodiments of embodiment 1-17, wherein the coacervates encapsulate a therapeutically effective amount of the compound; and administering the coacervates to the subject, thereby delivering the compound.

Embodiment 46: A method of treating obesity, the method comprising administering to a subject in need thereof, a therapeutically effective amount of the population of coacervates of any of the embodiments of embodiment 1-17, wherein the functional end group comprises catechol, and wherein the coacervates adhere to the gastrointestinal tract of the subject.

Embodiment 47: A method for treating an inflammatory bowel disease, the method comprising: providing the population of coacervates of any of the embodiments of embodiment 1-17, wherein the coacervates encapsulate a therapeutically effective amount of a compound for treating the inflammatory bowel disease, and wherein the functional end group comprises catechol; and administering the coacervates to the subject, thereby adhering at least a portion of the coacervates to the gastrointestinal tract of the subject and delivering the compound.

Embodiment 48: A composition comprising: the population of coacervates of any of the embodiments of embodiment 1-17; and a pharmaceutically acceptable excipient.

### EXAMPLES

The present disclosure will be better understood in view of the following non-limiting examples.

### Example 1

### Formation of Nanoparticle-Assembled Coacervates having Low Size Polydispersity via Hydrogen Bonding

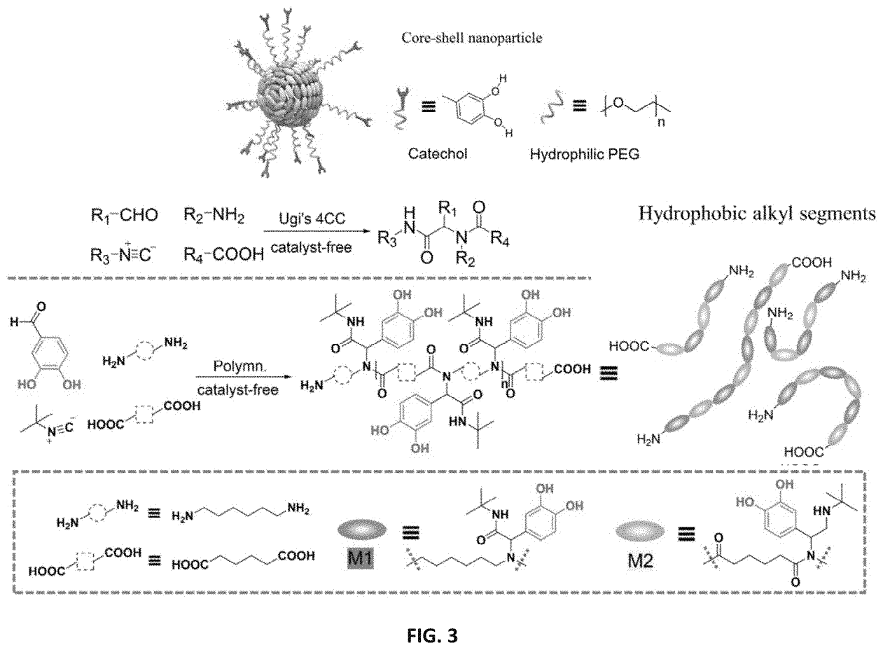

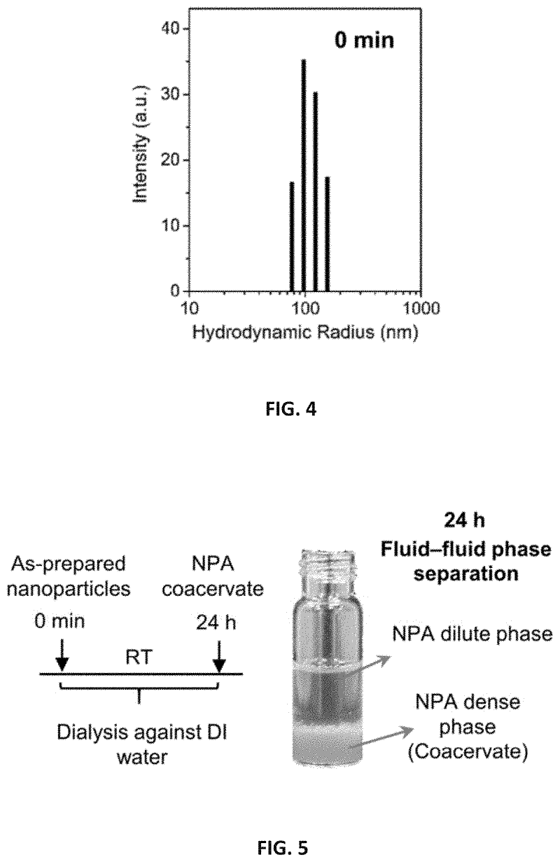

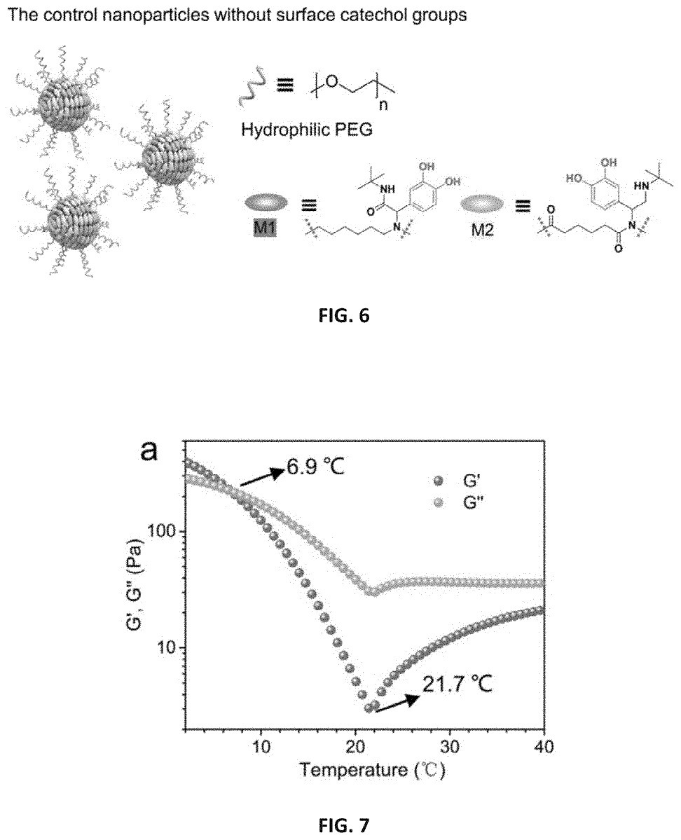

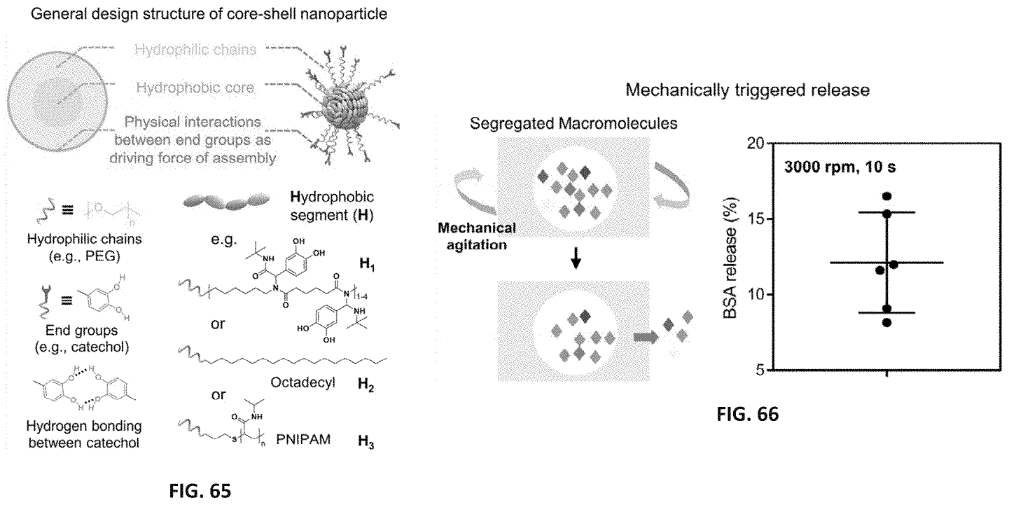

The core-shell nanoparticles displaying surface catechol groups (FIGS. 3 and 65) were first fabricated via the self-assembly of a synthesized amphiphilic polymer composed of the catechol-grafted hydrophilic PEG and hydrophobic alkyl segments. Dynamic light scattering (DLS) analysis confirmed the formation of as-prepared core-shell nanoparticles with a hydrodynamic radius around 100 nm (FIG. 4). The subsequent dialysis of core-shell nanoparticle suspension against deionized (DI) water under room temperature for 24 hours induced the in-situ assembly of nanoparticles, leading to the formation of dense phase vacuolated coacervate (referred to as Nanoparticle-Assembled or NPA coacervate thereafter) resulting from fluid-fluid phase separation (FIG. 5). Negative control nanoparticles without surface catechol groups failed to form the coacervate via the fluid-fluid phase separation (FIG. 6). This finding indicates the hydrogen bonding between the surface catechol groups of core-shell nanoparticles is critical to the self-assembly of core-shell nanoparticles and formation of coacervate (Ahn, Lee, Israelachvili, & Waite, 13 Nat. Mater. 867 (2014)).

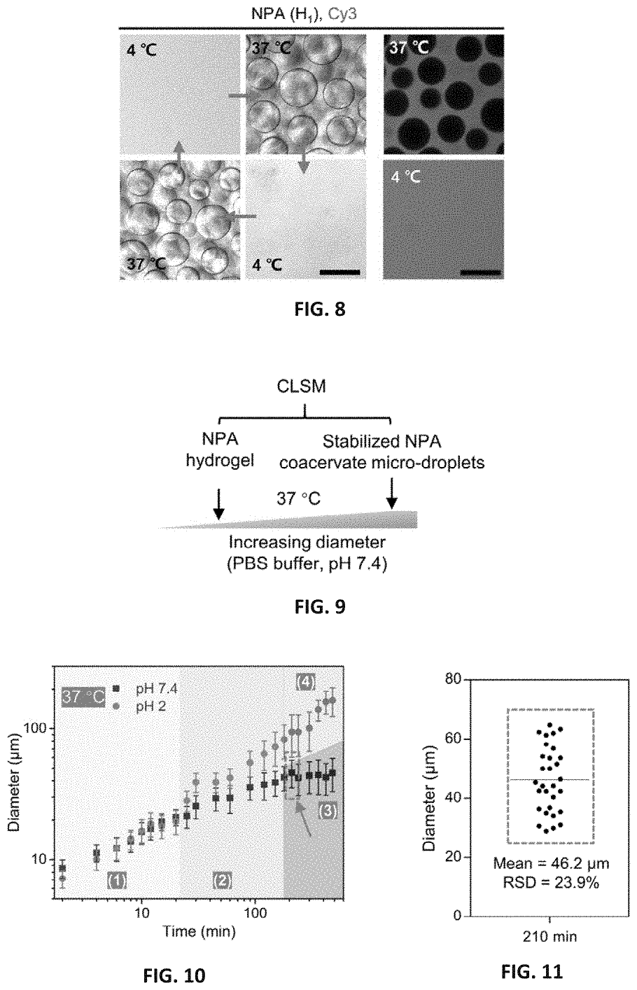

Rheological analysis showed the thermo-responsive properties of NPA coacervate (FIG. 7). The lowest storage modulus (G′) and loss modulus (G″) of NPA coacervate were detected at 21.7° C. Although the storage modulus (G′) increased with the temperature increasing to 40° C., the NPA coacervate remained liquid (G′ <G″). Interestingly, when the temperature decreased from 21.7° C. to 2° C., the liquid coacervate turned into a hydrogel (G′>G″), thus demonstrating an upper critical solution temperature (UCST) of around 6.9° C. Consistent with the rheological behavior, the formation of NPA coacervate and coacervate-to-hydrogel transition can be reversibly controlled by simply tuning the temperature between 4° C. (hydrogel) and 37° C. (coacervate) (FIG. 8). Therefore, the as-prepared NPA coacervate can be conveniently stored at 4° C. for a long period with no significant property change.

### Example 2

### Stabilization of Vacuolated Nanoparticle-Assembled Coacervate under Physiological Conditions

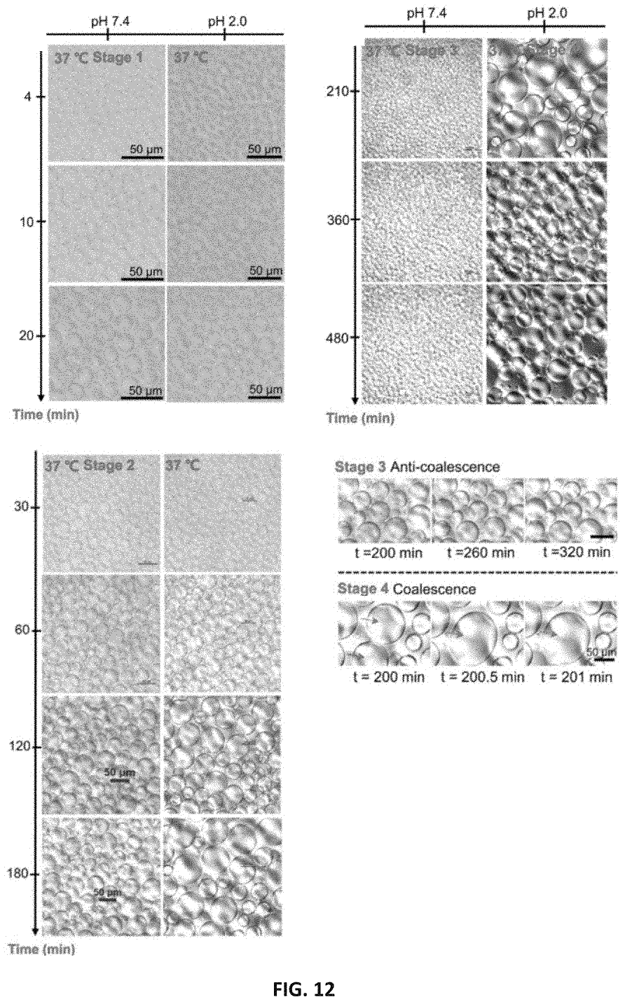

We next examined the stability of vacuoles in NPA coacervate under physiological condition at 37° C. in phosphate buffered saline (PBS) buffer by confocal laser scanning microscopy (CLSM) (FIG. 9). The diameter of vacuoles within NPA coacervate increased within the first 210 minutes at pH 7.4 (Stage 1 and 2) and remained almost unchanged for the following several hours (Stage 3), thus demonstrating the excellent anti-coalescence property (FIGS. 10 and 12). The average diameter of stabilized vacuoles was 46.2 μm with a relative standard deviation (RSD) of 23.9% (FIG. 11).

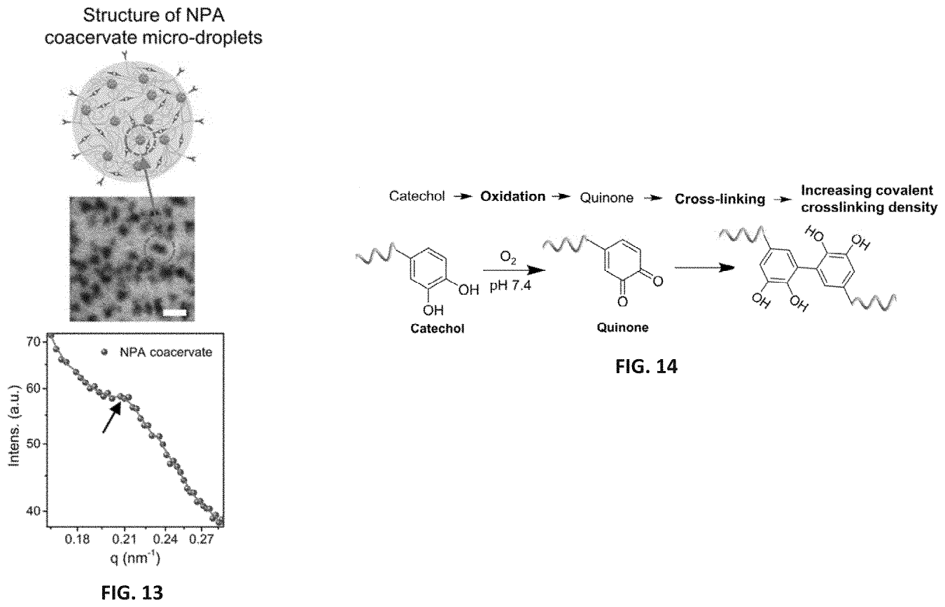

We further investigated the mechanism of the excellent anti-coalescence property of the vacuoles. Transmission electron microscopy (TEM) data confirmed the presence of core-shell nanoparticles in the vaculoated coacervate (FIG. 13). The dark spots in TEM images marked by red circle are believed to be the hydrophobic alkyl core of the assembled core-shell nanoparticle, while the hydrophilic PEG shell conjugated with surface catechol groups connect the hydrophobic alkyl cores to form stable NPA coacervate structures through catechol-mediated hydrogen bonding. Small angle X-ray scattering (SAXS) spectrum measurements of coacervates showed a broad correlation peak, which should be attributed to the random distribution of hydrophobic alkyl cores. By considering 0.21 nm⁻¹ as the peak position, the spacing between the hydrophobic alkyl cores was estimated as ˜30 nm, which was consistent with the TEM data.

Without being bound by a particular theory, it is believed that catechol-quinone oxidation under physiological condition gradually diminishes the hydrogen bonding between the surface catechol groups of NPA coacervate to limit their further growth, thereby stabilizing the vacuoles over time (Yang, M. Stuart, & Kamperman, 43 Chem. Soc. Rev. 8271 (2014); Barrett et al., 23 Adv. Funct. Mater. 1111 (2013)) (FIGS. 10 and 14 Stage 3). To test this hypothesis, vacuolated coacervate was incubated in pH 2 buffer, which protects the catechol groups from oxidation. Consistent with our hypothesis, the vacuoles continued to coalesce with each other and grew into larger droplets in pH 2 buffer even after several hours (FIGS. 10 and 14). These findings strongly indicate that the self-assembly of coacervate is driven by the surface catechol groups. Taken together, these results demonstrate that vacuolated bulk NPA coacervates can be stabilized under physiological environment without membranization, thus providing a stable and compartmentalized liquid microenvironment to mimic the natural cellular coacervates for regulating cell functions.

### Example 3

### Selective Restriction of Macromolecular Exchange by Resting State Coacervates

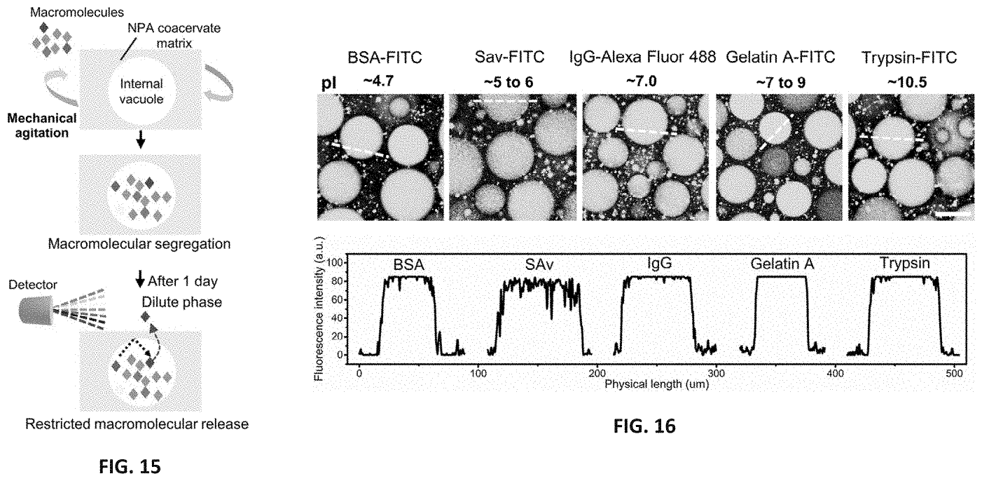

The macromolecular uptake by conventional complex coacervates is typically governed by the electrostatic parameters of macromolecules (McTigue & Perry, 15 Soft Matter 3089 (2019); Tang, Antognozzi, Vicary, Perriman, & Mann, 9 Soft Matter 7647 (2013)). Free of electrostatic interactions, the vacuolated NPA coacervate described in the previous Examples are a collection of core-shell nanoparticles assembled via hydrogen bonding and contain a layer of dense surface hydrophilic polyethylene glycol (PEG) chains. Therefore, we speculate that our NPA coacervate may have drastically different macromolecule uptake capability and mechanism (FIG. 15). We used proteins with an increased isoelectric point (pI) ranging from 4.7 to 10.5 to study the macromolecular uptake behaviors of the vacuolated NPA (H₁) bulk coacervate under mechanical agitation (FIG. 16). At pH values above or below the pI, the surface charge of a protein is negative or positive, respectively. Consistent with our hypothesis, confocal fluorescence images confirmed that mechanical agitation for just 10 seconds significantly enhanced the sequestration of all tested proteins into the vacuoles under the physiological conditions (PBS buffer, pH 7.4, FIG. 16).

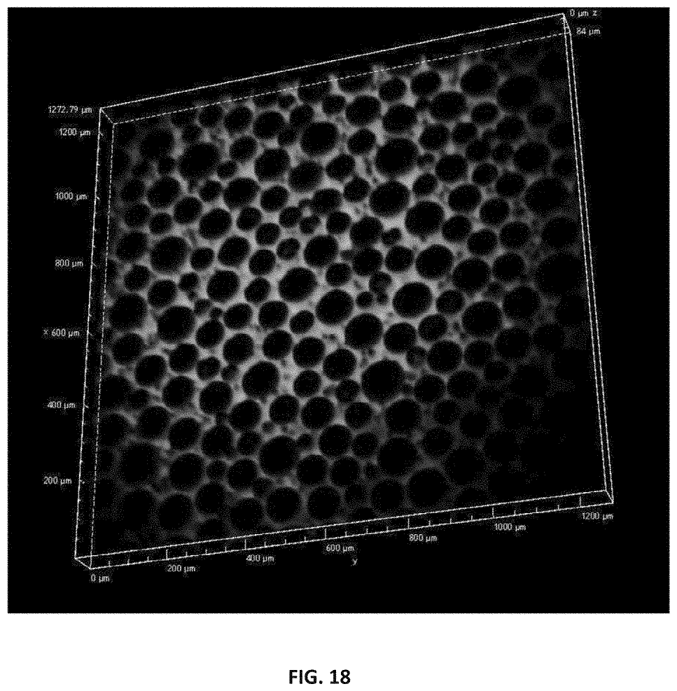

We next used BSA labeled with Texas Red or FITC as the model proteins to further evaluate the macromolecular exchange between the surrounding dilute solution, NPA (H₁) coacervate matrix (C), and the vacuoles (V, FIG. 19). Under the resting condition, the Texas Red-labeled BSA in the surrounding dilute solution barely infiltrated into the NPA coacervate matrix after 1 day, and so did the FITC-labeled BSA pre-loaded in vacuoles via mechanical agitation (FIG. 19). The semi-quantitative evaluation based on the fluorescence intensity profile further confirmed the limited diffusion of labeled BSA from both dilute solution and vacuoles to the coacervate matrix (FIG. 19). Furthermore, the infiltration of Texas Red-labeled BSA across NPA coacervate matrix (C) into vacuoles (V) was even more limited (FIG. 19). The mean fluorescent intensity (MFI) of Texas Red-labeled BSA in the coacervate matrix (Ic) was more around 28 times that of the vacuole (Iv) even after 1 day. 3D reconstruction of confocal images further confirmed the predominant localization of the limited amount of infiltrated BSA in the vacuoles. These findings indicate that the NPA coacervate matrix can significantly limit the diffusion of external BSA into the vacuoles under the resting condition.3D imaging of the BSA diffusion at day 1 also confirmed such heterogeneous spatial distribution of BSA (FIG. 18).

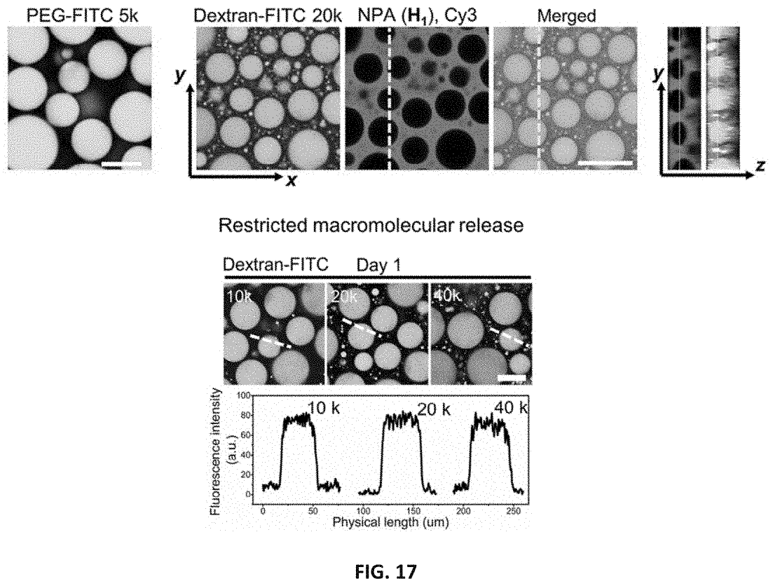

We next examined the capability of NPA coacervates to segregate non-proteinaceous macromolecules by using FITC-labeled synthetic polymer (PEG) or polysaccharide (Dextran) as the cargo molecule. Confocal fluorescence images revealed that both PEG-FITC (5 kDa) and dextran-FITC (20 kDa) were predominantly loaded in the vacuoles of NPA coacervate after mechanical agitation (FIG. 17). The 3D reconstruction of confocal images further confirmed that the dextran-FITC solution in the internal vacuoles was completely enclosed by the surrounding NPA (H₁) coacervate matrix labeled by the red fluorescent Cy3 dye (yz plane, FIG. 17). Additionally, varying the molecular weight of PEG (5, 10, 20, and 40 kDa) and dextran (10, 20, and 40 kDa) did not affect the successful loading and macromolecular segregation of these two polymers in the internal vacuoles of NPA coacervate (FIG. 17). Many previous studies reported the aqueous two-phase systems (ATPS) consisting of two water-soluble and yet incompatible components, such as PEG and dextran (Keating, 45 Acc. Chem. Res. 2114 (2012)). Although our NPA coacervates contain PEG as the hydrophilic component, the capability of the vacuolated NPA coacervates to segregate diverse macromolecules instead of only dextran indicates the distinct assembly mechanism and structural properties of NPA coacervates from that of ATPS.

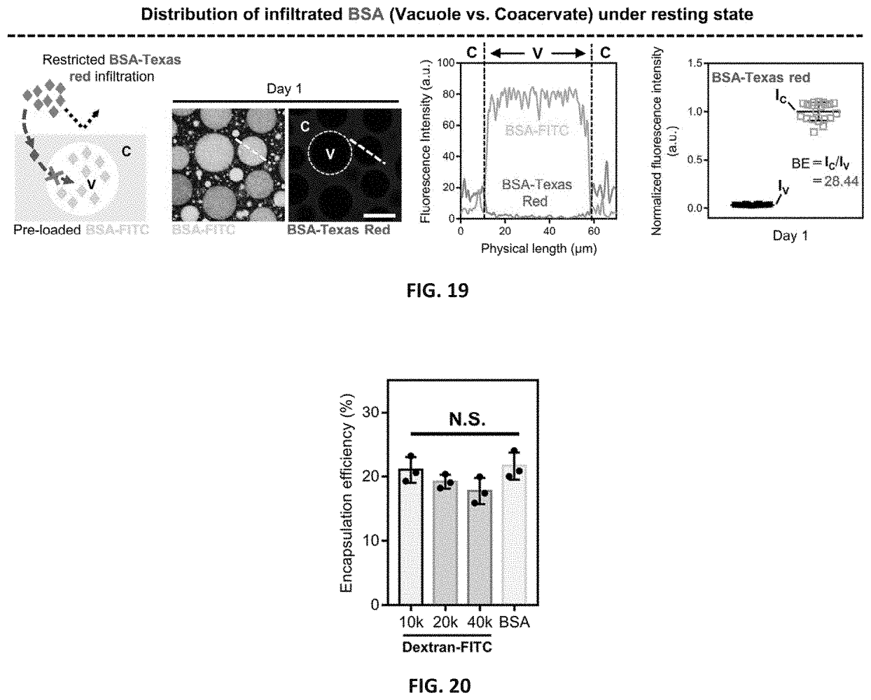

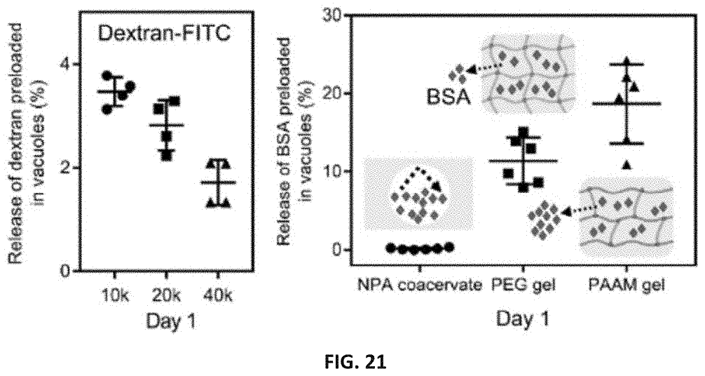

We also evaluated the capability of the NPA (H₁) coacervate matrix to limit the diffusional release of the macromolecules pre-loaded in the vacuoles. After 1 day of incubation, the majority of pre-loaded dextran-FITCs with different molecular weights of 10, 20, and 40 kDa remained in the vacuoles (FIG. 20). No significant difference was found between the encapsulation efficiencies of diverse dextran-FITCs and BSA, further indicating that the loading of external macromolecular cargoes into the vacuoles of NPA coacervates is driven by the physical force derived from the mechanical agitation rather than the supramolecular interactions between the NPA coacervate matrix and diverse macromolecular cargoes. Only small fractions (1-3%) of dextran were detected in the surrounding dilute solution (FIG. 21). Therefore, the NPA (H₁) coacervate at least provides a segregated environment for macromolecules larger than 10 kDa dextran. Additionally, less than 1% of the pre-loaded BSA was released from the NPA (H₁)coacervate after 1 day, whereas the conventional permeable hydrogels with similar solid contents released 10-20% of the pre-loaded BSA during the same period (FIG. 21). These findings indicate that vacuolated NPA coacervates can maintain the segregation of the pre-loaded macromolecules by restricting the diffusional release. Furthermore, beacuse mechanical agitations can rupture the vacuole wall before the self-healing of liquid NPA caocervates closes the vacuoles again when the agitations end, applying mechanical agitation (vortex for 10 s at 3000 rpm) can trigger a brief burst release (12.12±3.316%) of pre-loaded BSA from the NPA coacervates incubated in fresh buffer (FIG. 66). Consistent with the finding from the NPA (H₁)coacervate, NPA (H₂)and (H₃)coacervate can also load and segregate macromolecules (e.g., dextran) in the internal vacuoles. This finding demonstrates that this strategy of preparing vacuolated NPA coacervates based on the in situ self-assembly of core-shell nanoparticles for regulating macromolecule distribution is generalizable to nanoparticles with diverse chemical makeups.

### Example 4

### Activation of Macromolecular Uptake by Vacuolated Coacervates by Mechanical Agitation

We next studied the macromolecular uptake of the vacuolated coacervate under mechanical agitation (FIGS. 15-17). The uptake efficiency (UE) can be calculated as the reciprocal of BE, i.e., by dividing the MFI in the coacervate compartments (In) by the MFI in the surrounding (Out). Therefore, a larger UE value means a better uptake efficiency of macromolecules by the vacuolated coacervate. Mechanical stirring for 10 seconds significantly enhanced the diffusion of all tested macromolecules into the vacuolated coacervate. These results show that the restricted macromolecular diffusion into the vacuolated coacervate under resting condition can be disrupted by mechanical agitation. Under the resting condition, the densely-assembled nanoparticles (Koga, Williams, Perriman, & Man, 3 Nat. Chem. 720 (2011); Yin et al., 7 Nat. Commun. 10658 (2016)) with the hydrophobic core and dense PEG chains (Keating, 45 Acc. Chem. Res. 2114 (2012)) create a highly molecularly-crowded environment in the vacuolated coacervate, thus effectively restricting the macromolecular diffusion (Zustiak, Nossal, & Sackett, 101 Biophys. J. 255 (2011); Clague & Phillips, 8 Phys. Fluids 1720 (1996)). The applied mechanical forces can transiently disrupt the structure of vacuolated coacervate and create a window of opportunity for macromolecules to be captured and concentrated by the coacervate through hydrogen bonding and hydrophobic interaction. The diffusion and biotransport of macromolecules produced by cells is essential to the regulation of diverse cellular functions via the autocrine, paracrine and endocrine mechanism. The unique macromolecular barrier/enrichment capability of the vacuolated NPA coacervate makes it an ideal 3D compartmentalized cell microenvironment with spatiotemporal molecular heterogeneity to study cellular development in such controlled environment.

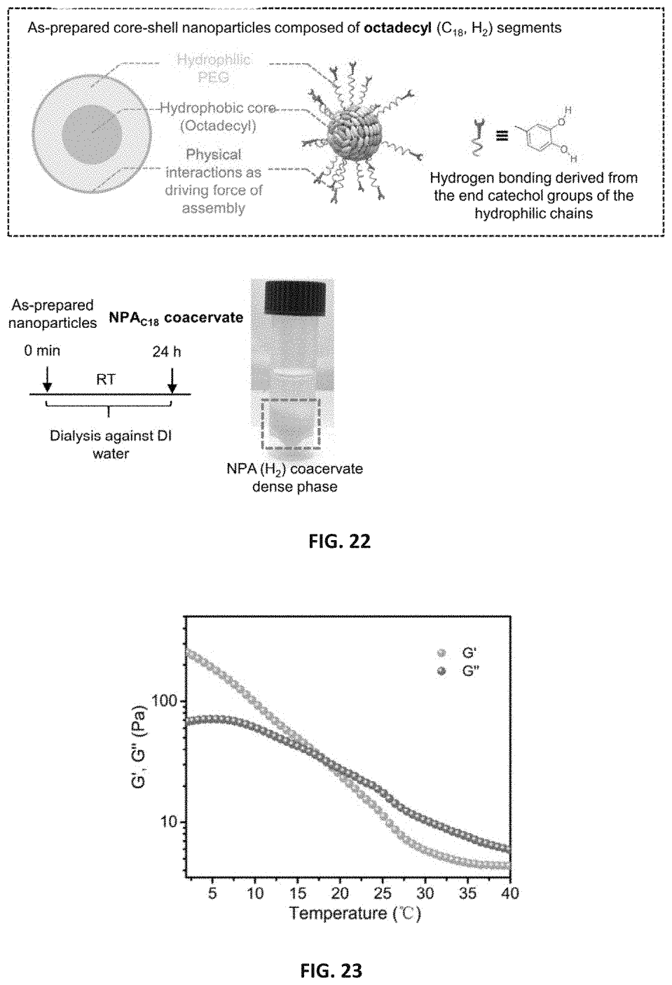

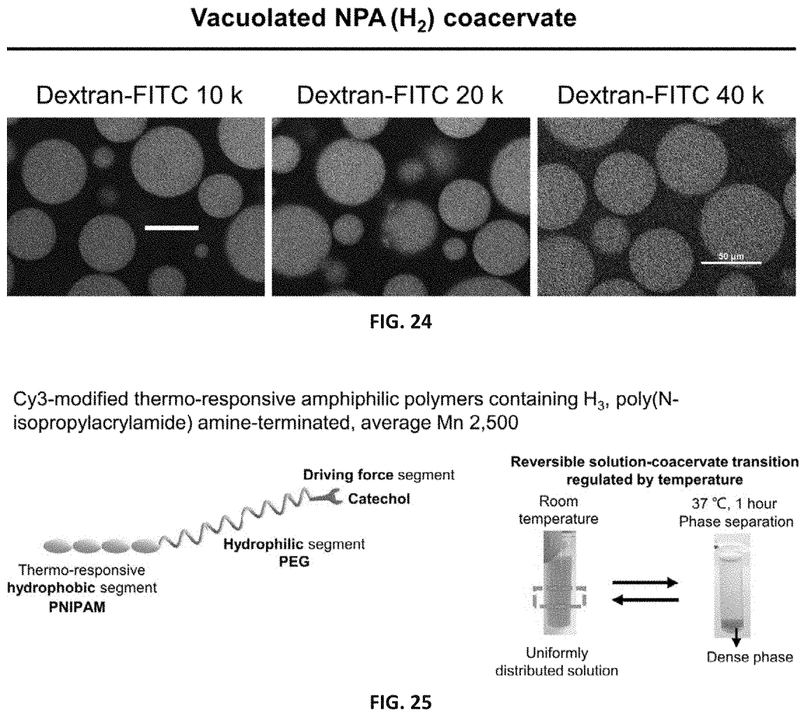

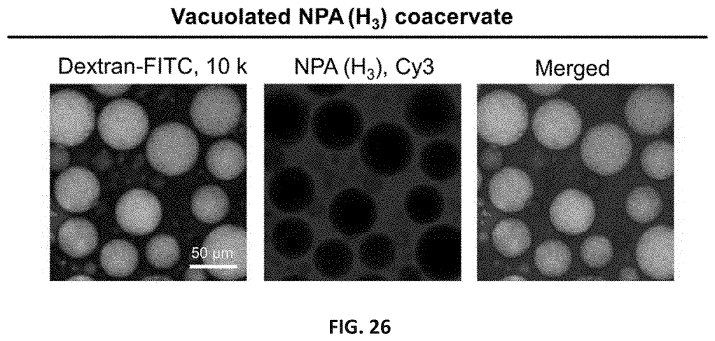

To further verify the mechanism and versatility of our strategy for preparing the NPA coacervates, the hydrophobic cores of core-shell nanoparticles were replaced by the commercially available octadecyl (C18, H₂) group (FIG. 22) or thermo-responsive poly(N-isopropylacrylamide) (PNIPAM, H₃). The dense NPAC18 vacuolated coacervate was formed successfully via the fluid-fluid phase separation. The rheological analysis of NPAC18 coacervate showed a similar hydrogel-coacervate transition behavior (at 18.16° C.) as that of the NPA coacervate (FIG. 23). Also, upon switching to a temperature above the lower critical solution temperature of PNIPAM (LCST, ˜32° C.), the nanoparticles containing the PNIPAM (H₃)as the hydrophobic core also assembled into the bulk NPA (H₃)coacervate. These findings demonstrate that the provided strategy of preparing NPA coacervate based on in-situ self-assembly of core-shell nanoparticles for regulating macromolecule transport is generalizable to diverse nanoparticle chemical makeups.

### Example 5

### Regulation of Cell Behaviors through Molecular Heterogeneity

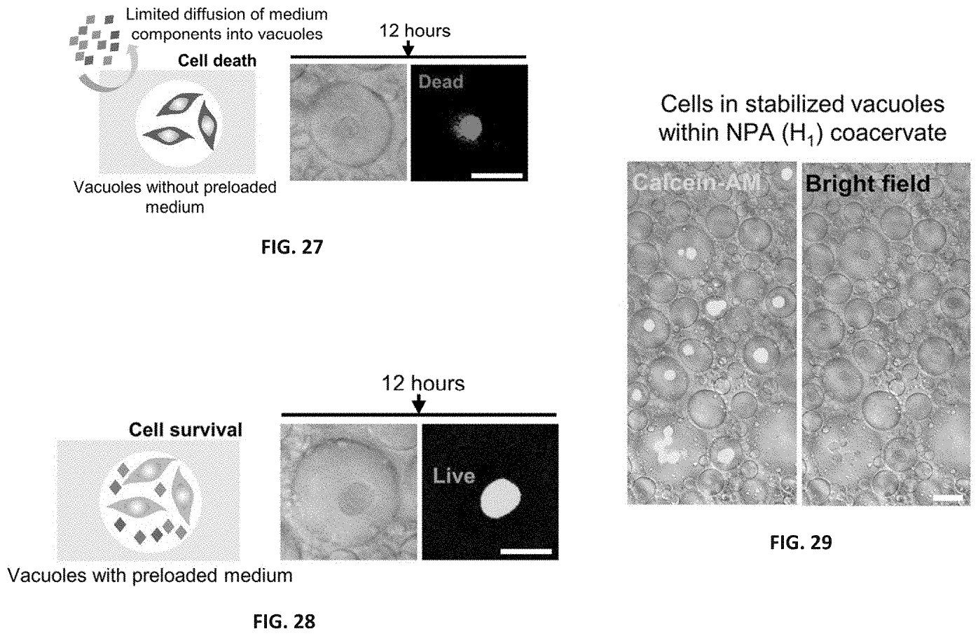

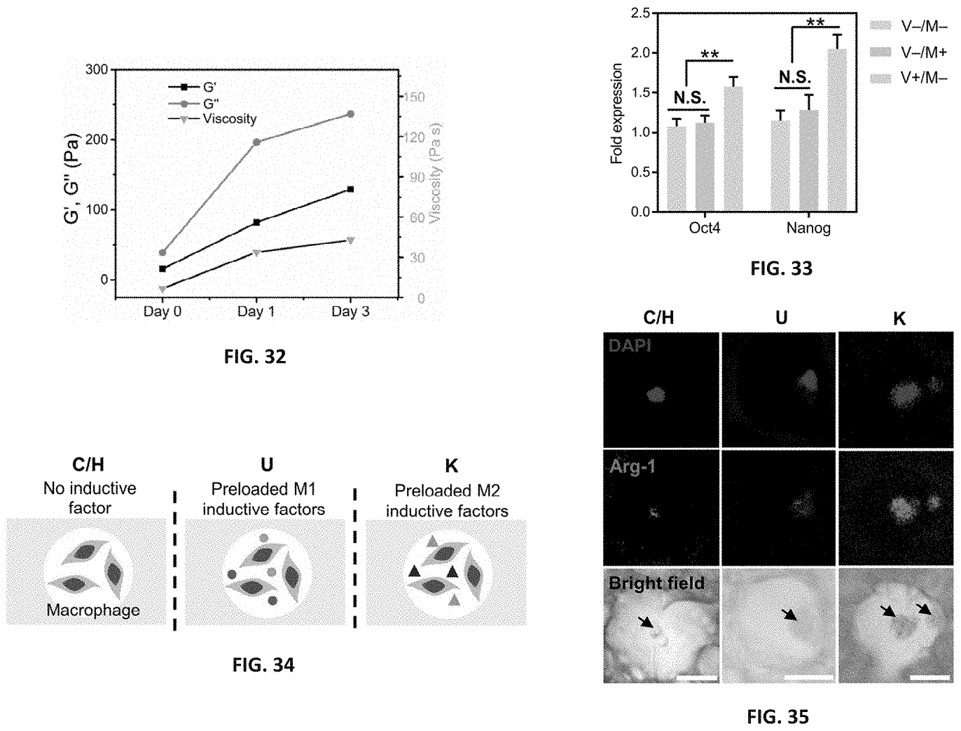

To verify the feasibility for 3D cell culture, vacuolated NPA coacervate was mixed with fetal bovine serum (FBS)-supplemented medium via mechanical agitation to preload the FBS into coacervate. Hela cells were labelled with the live cell dye Calcein-AM before being encapsulated in the vacuolated coacervate with or without FBS preloading by gentle mixing of cell suspension and coacervate, respectively (FIGS. 27 and 28). Confocal laser scanning microscopy (CLSM) images confirmed that the cells were well encapsulated in the vacuolated NPA coacervate (FIG. 29). The cell-laden vacuolated coacervate was subsequently cultured in FBS-supplemented medium under resting condition for 1 day before staining the dead cells with red fluorescence by propidium iodide. The cells in the vacuolated coacervate without preloaded FBS were almost all dead after 1 day of culture in coacervate due to the restricted influx of medium FBS into coacervate during culture (FIG. 27). In contrast, most cells in the vacuolated coacervate with preloaded FBS remained predominantly viable (more than 90%) in the coacervate because the preloaded FBS prevented cells from starvation (FIG. 28). Long-term monitoring of the rheological properties of NPA coacervate under cell culture condition showed no change in the coacervate state (G″>G′) for up to 5 days despite a slight increase in both shear moduli (FIG. 32). These findings not only further confirm the capability of NPA coacervate to limit macromolecular exchange with the outside environment but also demonstrate the feasibility of NPA coacervate to potentially support long-term 3D cell culture.

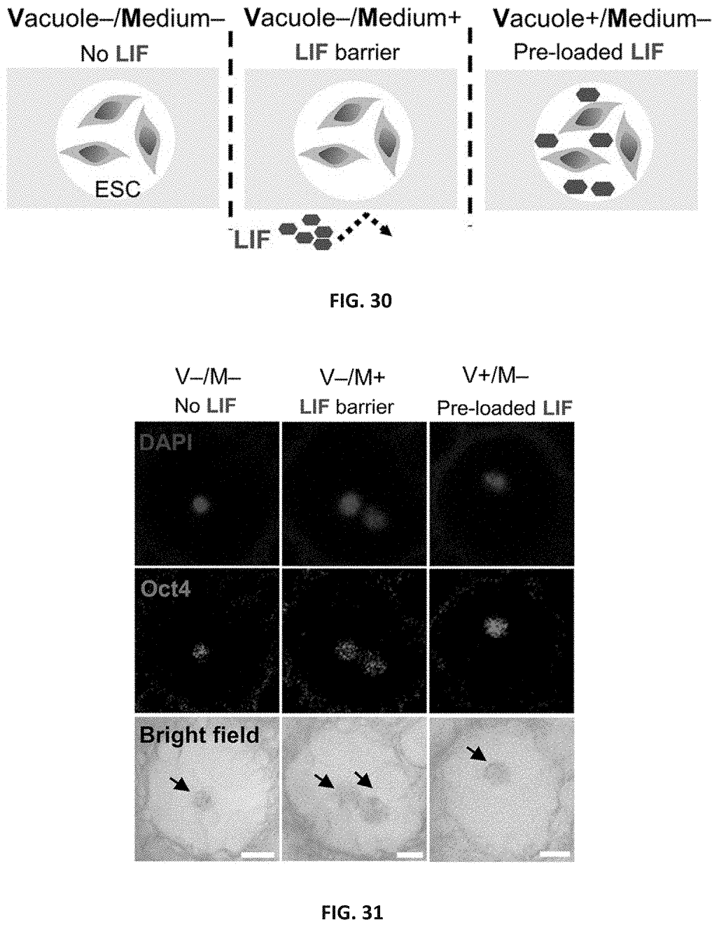

We next evaluated the efficacy of NPA coacervate to support the macromolecular supplement-dependent functions of encapsulated cells. We encapsulated mouse embryonic stem cells (mESCs) into the vacuolated NPA coacervate, which was preloaded with or without the leukemia inhibitory factor (LIF), an essential supplement for maintaining the mESC pluripotency (FIG. 30) (Huang, Yan, Ye, Tong, & Ying, 32 Stem Cells 1149 (2014); Casanova et al., 29 Stem Cells 474 (2011)). The mESCs in the coacervate without LIF preloading exhibited diminishing nuclear presence of pluripotency marker October 4 after 36 hours of culture in both blank (In−/Out−) and LIF-supplemented medium (In−/Out+) (FIG. 31). The symbols−/+ indicate the absence and presence of LIF in the coacervate (In) or medium (Out), respectively. In contrast, the mESCs in the coacervate with LIF preloading maintained the high level of nuclear October 4 despite being culture in LIF free medium (In+/Out−) (FIG. 31). Consistent with immunofluorescence staining results, quantitative reverse transcription polymerase chain reaction (qPCR) data also revealed the significantly higher expression of October 4 and another pluripotency marker, Nanog, by mESCs in the In+/Out− group compared with the two control groups (FIG. 33).

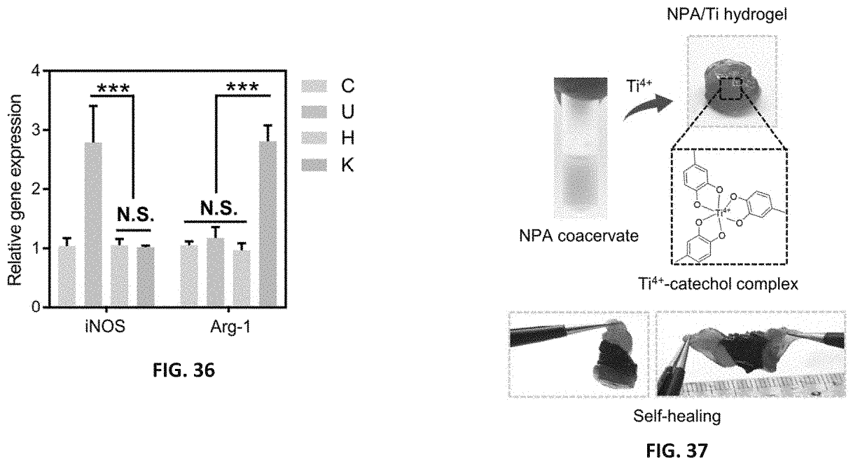

We further evaluated the macromolecular barrier function of NPA coacervate to support differential cell differentiation in the same culture environment. Mouse macrophages, which can differentiate into the M1 pro-inflammatory or M2 pro-healing phenotype, were encapsulated in vacuolated coacervates, which were further patterned into alphabetic letters “C, U, H, and K” and cultured in basal culture medium for 24 hours (FIG. 34) (Kang et al., 10 Nat. Commun. 1696 (2019); Murray & Wynn, 11 Nat. Rev. Immunol. 723 (2011); Lawrence & Natoli, 11 Nat. Rev. Immunol. 750 (2011)). Letters “U” and “K” were pre-loaded with M1 and M2 inductive factors, respectively, while letters “C” and “H” were not loaded with any factors. Immunofluorescence staining against M1 marker (iNOS) or M2 marker (Arg-1) revealed that the macrophages in “U” and “K” patterns had the highest expression of iNOS and Arg-1, respectively (FIG. 35). In contrast, macrophages in “C” and “H” patterns were not polarized into neither M1 nor M2 phenotype. The VCR results further confirmed that the M1 and M2 inductive factors preloaded in “U” and “K” alphabets did not induce the polarization of macrophages in “C” and “H” alphabets (FIG. 36). These findings indicate that the vacuolated NPA coacervate limited the outward diffusion of M1/M2 inductive factor from the loaded alphabets into unloaded alphabets despite sharing the same pool of culture medium. In other words, the NPA coacervate can establish and maintain the spatial macromolecular heterogeneity to modulate differential supplement-dependent cell functions in a common liquid culture environment.

### Example 6

### Alteration of Macromolecular Permeability by Coacervate-Hydrogel Transition

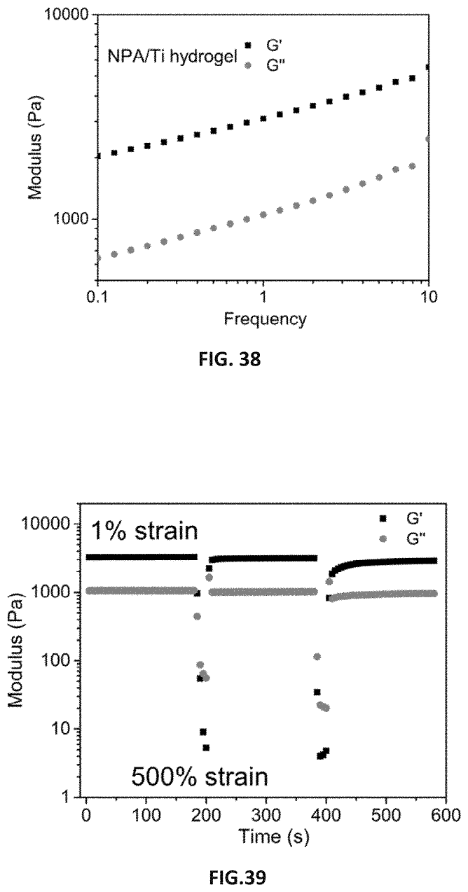

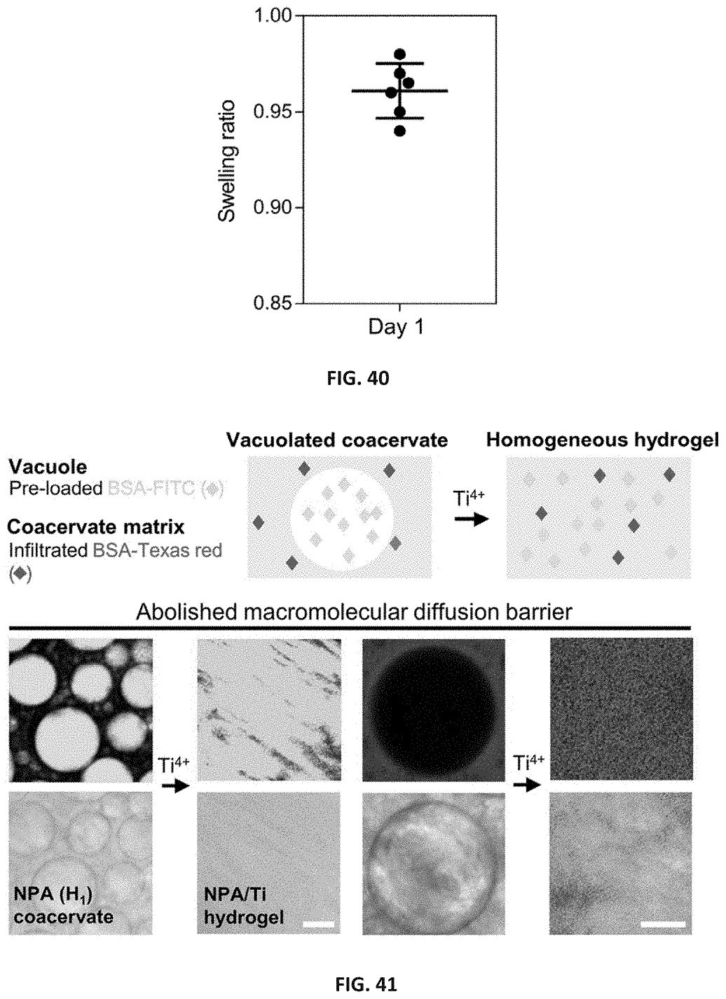

We have demonstrated the mechanical agitation-induced macromolecular uptake by the NPA coacervate. The enhanced macromolecular exchange of the coacervate with outside environment can also be achieved by transforming NPA coacervate into hydrogels, which are known to have a highly permeable 3D network. Coacervate-hydrogel transition is not rare in cells (Shin & Brangwynne, 357 Science eaaf4382 (2017)). For example, the coacervates formed by RNA-binding proteins exhibit liquid metastability and can transform into hydrogels composed of amyloid-like fibers (M. Kato et al., 149 Cell 753 (2012); Murakami et al., 88 Neuron 678 (2015)). At pH 7.4, the addition of Ti⁴⁺ turned the NPA coacervate into a self-healing NPA/Ti hydrogel crosslinked by Ti⁴⁺—catechol coordination (FIG. 37). Cut NPA/Ti hydrogel pieces can self-heal into one complete hydrogel (the middle NPA/Ti hydrogel was stained for better visualization) (FIG. 37). The rheological analysis further confirmed the successful coacervate-hydrogel transition. Due to the dynamic nature of Ti⁴⁺—catechol coordination, the NPA/Ti hydrogels showed frequency-dependent storage (G′) and loss (G″) moduli with G′ being higher than G″ (FIG. 38). Moreover, the alternative high/low shear loading revealed the shear-thinning properties of the NPA/Ti hydrogel under high shear and immediate self-healing upon switching to low shear (FIG. 39). Furthermore, after the coacervate-hydrogel transition NPA/Ti hydrogel showed no significant swelling after incubation in PBS buffer (37° C.) for 3 days likely due to the combination of the strong catechol-Ti⁴⁺ coordination bond and hydrophobic alkyl core of the assembled nanoparticles (FIG. 40). The non-swollen NPA/Ti hydrogels provide a stable culture microenvironment for 3D cell culture (Kamata, Akagi, Kayasuga-Kariya, Chung, & Sakai, 343 Science 873 (2014)).

To evaluate the macromolecule diffusion after the coacervate-hydrogel transition, we pre-loaded FITC-labeled BSA and Texas Red-labeled BSA in the vacuoles and coacervate matrix, respectively (FIG. 41). After the coacervatehydrogel transition triggered by Ti⁴⁺, uniform distributions of both FITC and Texas Red-labeled BSA proteins were found within the NPA/Ti hydrogel network. The analysis of cell metabolic activity via MTT assay as an indicator of cell viability confirmed the cytocompatibility of this coacervate-hydrogel transition. These findings demonstrate that the coacervate-hydrogel transition can abolish the restriction on the macromolecule diffusion by the NPA coacervate on demand in a cytocompatible manner.

### Example 7

### Reduction of Gastrointestinal Tract Nutrient uptake with Bioadherant Coacervates

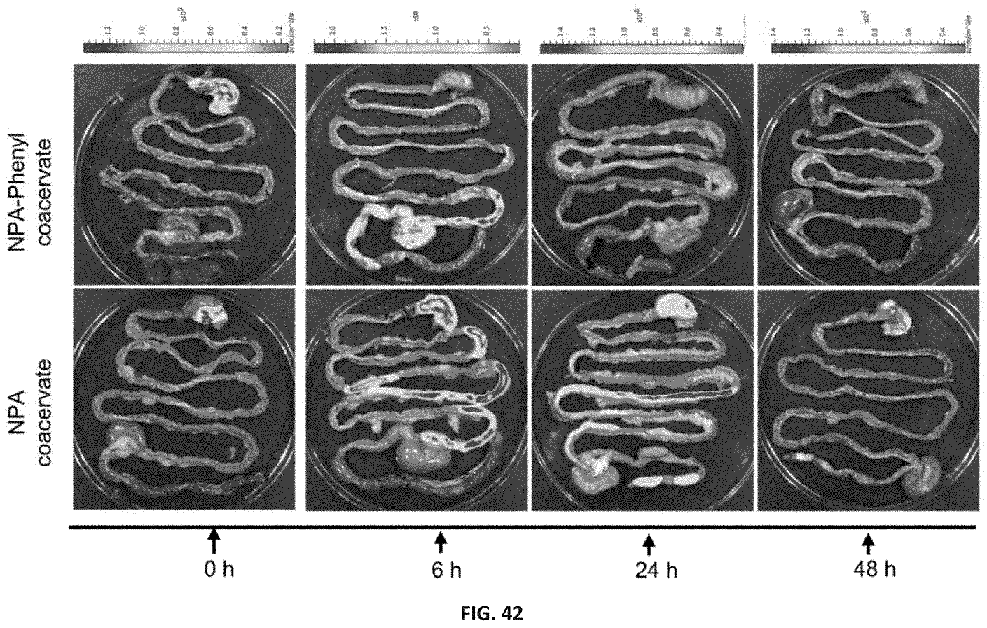

After verifying the ability of NPA coacervate to control macromolecular spatiotemporal distribution, we further evaluated the efficacy of NPA coacervate to mediate sustained drug release. After gavage, NPA coacervate (modified with Cy7 tag) adhered to the gastrointestinal tract (GI tract) of rats for at least 48 hours, whereas the control NPA-phenyl coacervate showed much weaker adhesion (FIG. 42). Therefore, the prolonged retention of NPA coacervate in GI tract is ascribed to the catechol groups, which enhance the bioadhesion of NPA coacervate. In contrast, NPA-Phenyl coacervate, which contains no catechol groups, showed much weaker bioadhesion ability.

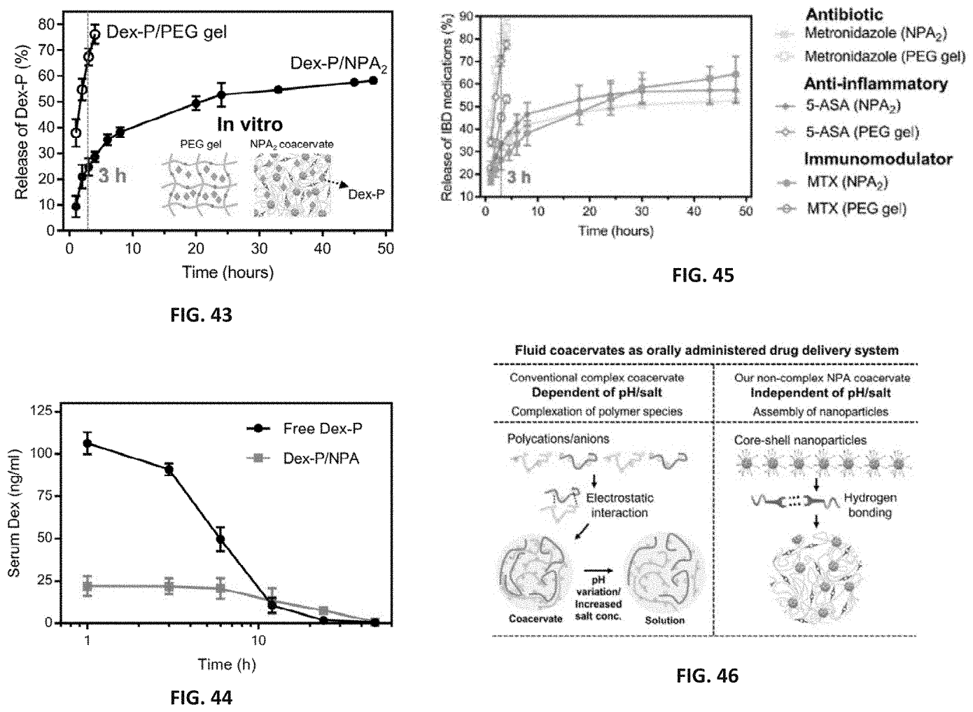

Lack of controlled drug release may result in significant systemic side effects. Our NPA coacervate showed the prolonged release of pre-loaded dexamethasone sodium phosphate (Dex-P), a water-soluble prodrug of Dex, under both in vitro (FIG. 43) and in vivo condition (FIG. 44). The plasma concentration of Dex in rats spiked at 1 hour after gavage of the Dex-P aqueous solution and decreased rapidly (Free Dex-P), whereas the plasma Dex concentration in the rats receiving gavage of Dex-P-laden NPA coacervate remained consistently at a lower level for over 24 hours (FIG. 44). The total dosage of Dex-P administered in the two groups was the same. Therefore, the provided NPA coacervate can mediate controlled and sustained release of the preloaded water-soluble drug molecules.

### Example 8

### Mediation of Sustained Drug Release in the GI Tract using NPA Coacervate

To demonstrate the versatility of NPA coacervate to mediate sustained drug release, other first-line small molecule drugs used to treat IBD including antibiotic metronidazole (Metro), anti-inflammatory 5-aminosalicylic acid (5-ASA), and immunoregulatory methotrexate disodium salt (MTX) were also encapsulated into the NPA coacervate to investigate the release kinetics. The NPA coacervate showed high encapsulation efficiency and prolonged release kinetics of these drugs, especially when compared with the burst release kinetics of PEG hydrogels (FIG. 45). In addition, the dried drug-laden NPA coacervate that is desirable for oral administration could be prepared via lyophilization, and simply adding dried drug-laden NPA coacervate into simulated gastric fluid or water could realize the rehydration to form fluid NPA coacervate with sustained-release kinetics of diverse drugs similar to the freshly prepared drug-laden NPA coacervate before lyophilization.

### Example 9

### Assembly of Core-Shell Nanoparticles into Non-Complex Coacervate to Adapt to the Harsh GI Tract Environment

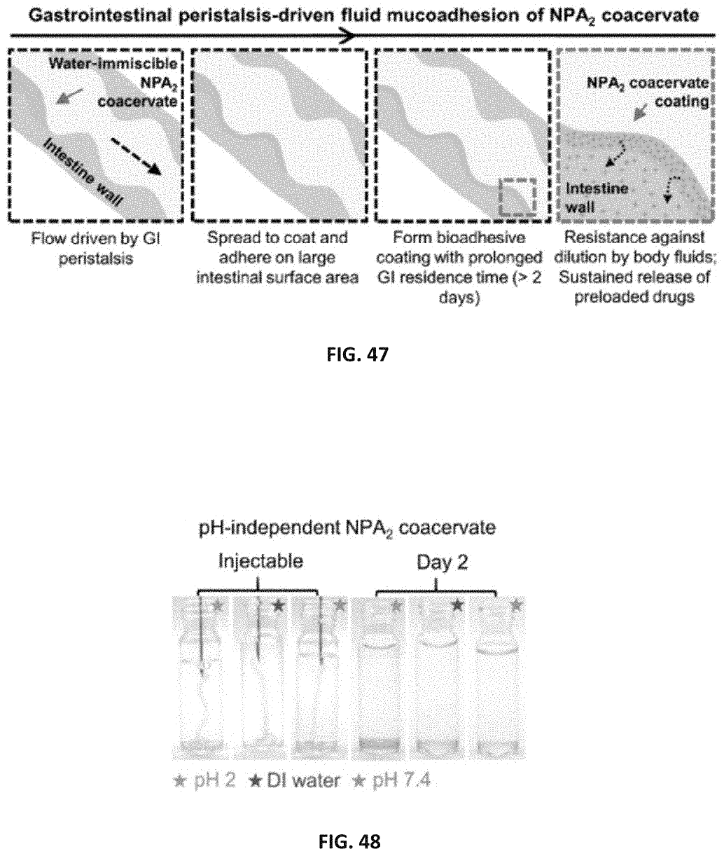

Considering that the GI tract routinely undergoes substantial changes in motility, fluid content, and acidity (from pH 1.5 in the stomach to pH 6.15-7.88 in intestines) (Vass et al., 296 J. Control. Release 162 (2019); Khutoryanskiy, 14 Nat. Mater. 963 (2015)), conventional complex coacervates can be easily disrupted by pH/salt variations (FIG. 46) (Love et al., 132 Angew. Chem. 6006 (2020); Wang & Schlenoff, 47 Macromolecules 3108 (2014); Chang et al., 8 Nat. Comm. 1 (2017)). In contrast, the provided water-immiscible, bioadhesive, and non-complex liquid coacervates derived from hydrogen bonding-driven self-assembly of nanoparticles can be more advantageously suitable for use as an orally administered intestinal-coating formulation (FIG. 46). Driven by the gastrointestinal peristalsis, the NPA coacervate can effectively spread to coat and adhere on large intestinal surface area with prolonged residence time of more than 2 days to mediate sustained release of loaded drugs (FIG. 47).

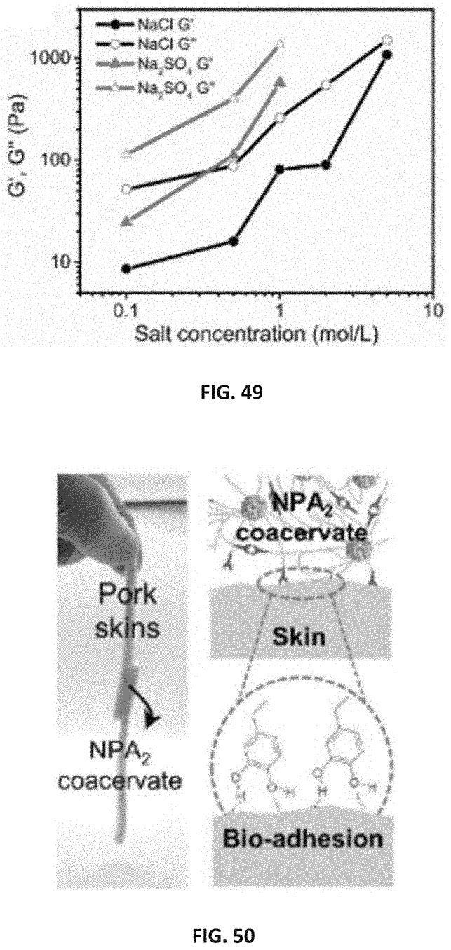

Free from electrostatic interactions, the provided NPA coacervate can be pH- and salt-independent. Compared with conventional complex coacervates that depend on pH values, the NPA coacervate remained stable after 2 days and did not become a single-phase solution under a wide range of pH conditions (FIG. 48). Furthermore, the NPA coacervate exhibited a salting-out effect (Yang, Wang, Yang, Shen, & Wu, 28 Adv. Matr.7178 (2016); He, Huang, & Wang, 28 Adv. Funct. Mater. 1705069 (2018)), and the shear moduli (G′ and G″) and viscosity increased with increasing salt concentrations (FIG. 49). It should be noted that electrostatic interactions between polyanions and polycations weaken with increasing salt concentrations; therefore, a critical salt concentration of 0.8-2.0 M NaCl generally can lead to the rapid dissociation of complex coacervates (Sing & Perry, 16 Soft Matter 2885 (2020); Wang & Schlenoff, 47 Macromolecules 3108 (2014)). However, the provided NPA coacervate remained as a viscous liquid (G′<G″) in 5.0 M NaCl, further confirming that the non-complex NPA coacervation should be attributed to hydrogen bonding-induced nanoparticle assembly rather than electrostatic interactions.

### Example 10

### Wet and Digestion-Resistant Characteristics of Biocompatible and Fluid NPA Coacervate

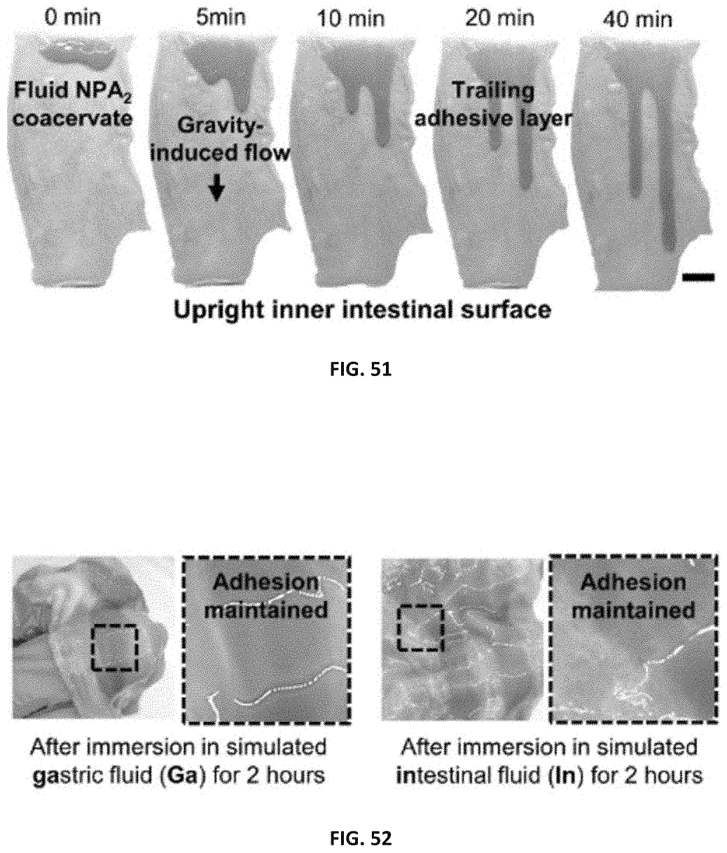

The catechol-mediated wet bioadhesion of NPA coacervate was sufficiently strong to glue two ribbons of the pork skin tissue together and hold the tissue weight (FIG. 50). The adhesive energy (G_{ad}) of NPA coacervate was estimated to be ˜7.07 J m⁻², similar to the previously reported values (around 2-10 J m⁻²) for nanoparticle-based (Rose et al., 505 Nature 382 (2014)) and polymeric adhesives (Zhao et al., 8 Nat. Comm. 2218 (2017); Liu, Tan, & Scherman, 130 Angew. Chem. 8992 (2018)). We next investigated the influence of the physical peristalsis and chemical environment (gastric acid and intestinal fluid) of the GI tract on NPA coacervate coating by using simulated ex vivo experiments. When deposited on the upright intestinal mucosa surface, the fluid NPA coacervate can adhere to the fresh and wet mucosa and steadily flow down driven by gravity, leaving a trailing adhesive coating layer (FIG. 51). After soaking the NPA coacervate-coated intestinal mucosa tissues in simulated gastric fluid (Ga) or simulated intestinal fluid (In) at 37° C. for 2 hours, respectively, the adherent coacervate coatings remained undiluted and maintained adhesion on the mucosa surface (FIG. 52).

### Example 11

### Enhanced Therapeutic Efficacy of DEX-P-Laden NPA Coacervate in a Rat Model of DSS-Induced Colitis

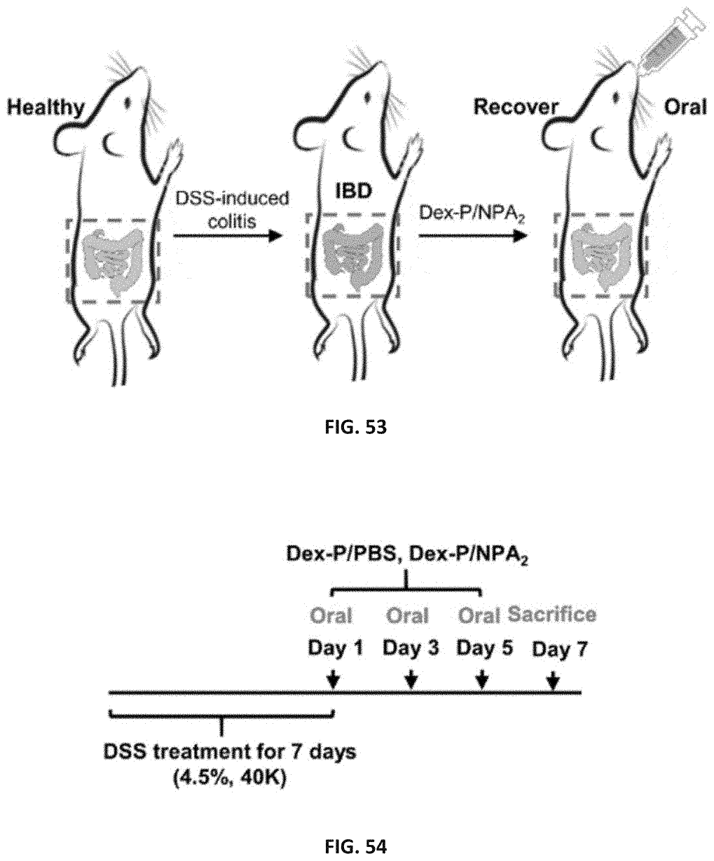

We next evaluated the therapeutic efficacy of a Dex-P-laden NPA coacervate in a rat model of dextran sulfate sodium (DSS)-induced colitis (FIG. 53). SD rats weighing around 250 g were given 4.5% DSS in drinking water for 7 days to develop acute colitis. Clinical manifestations of the colitis, such as severe rectal bleeding, watery diarrhea, and colonic edema were observed after 7 days. After successfully establishing the colitis model in rats, colitic SD rats received oral gavages of Dex-P-laden NPA coacervates (Dex-P/NPA) or the equivalent amount of Dex-P in PBS (Dex-P/PBS) on days 1, 3, and 5 (FIG. 54). Untreated colitic SD rats were used as the negative control. All SD rats were allowed unrestricted access to water and standard laboratory diet before and after oral gavage and sacrificed on day 7 for further evaluation of colon weight and length, histological severity, IBD-associated colonic myeloperoxidase (MPO)-activity, mRNA levels of tight junction-associated proteins (ZO-1 and occludin-1) and pro-inflammatory cytokines, such as interleukin IL-1β and tumor necrosis factor (TNF) in the distal colon.

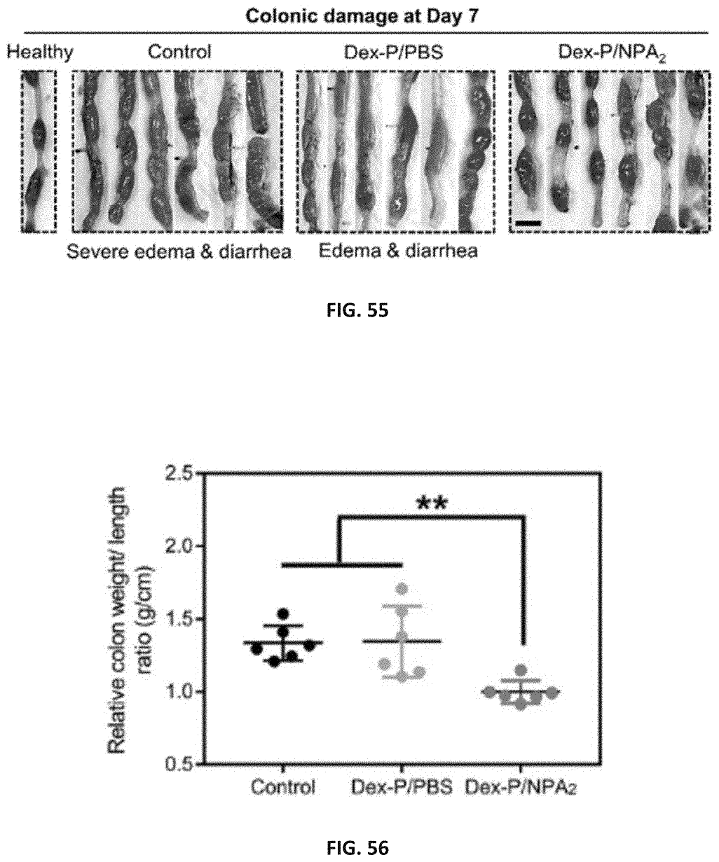

Our results demonstrated the significant therapeutic efficacy of Dex-P/NPA treatment against DSS-induced acute colitis. Dex-P-laden NPA coacervate significantly alleviated colonic edema and diarrhea caused by DSS-induced acute colitis (FIG. 55). Relieved edema in colitic SD rats receiving Dex-P/NPA was further confirmed by the lower colon weight/length ratio (FIG. 56). Compared to colitic SD rats receiving Dex-P solution in PBS (Dex-P/PBS), Dex-P/NPA treatment effectively protected SD rats against DSS-induced shortening of colon length.

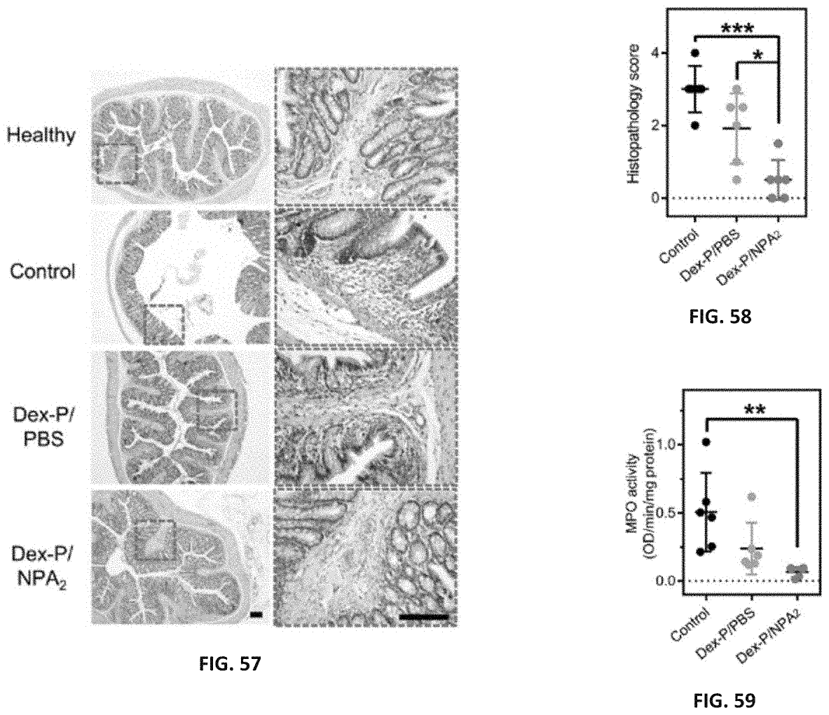

Representative images of hematoxylin and eosin (H&E) staining demonstrated significantly reduced histological inflammation in the colitic SD rats receiving Dex-P/NPA, while histological damages, such as the compromised integrity of the mucosal epithelial lining, decrease in villus height and crypt depth, interstitial edema and inflammatory infiltration, were observed in untreated colitic SD rats (Control) or rats treated with the equivalent amount of Dex-P solution in PBS (Dex-P/PBS, FIG. 57). Furthermore, histopathology scoring of H&E-stained tissue sections was used to evaluate the severity of colonic histological damage in a blinded fashion by a trained pathologist. Disease severity in colitic SD rats receiving Dex-P/NPA decreased significantly (mean histopathology score, 0.500) compared with colitic SD rats in the Dex-P/PBS group (mean histopathology score, 1.917) and the untreated control group (mean histopathology score, 3.000). Histopathology scores of colitic SD rats in the Dex-P/PBS group were not significantly different from the untreated control group (P=0.056, FIG. 58).

Colonic MPO activity in colitic SD rats receiving Dex-P/NPA was also significantly reduced compared with the untreated control group (FIG. 59) (Wilson et al., 9 Nat. Mater. 923 (2010)). Although we also observed a reduction of MPO activity in the Dex-P/PBS group due to the therapeutic activity of Dex-P against IBD, the high serum Dex level associated with such administration of Dex-P aqueous solution indicates an increased risk of complications related to severe systemic drug exposure (FIG. 58). Taken together, oral delivery of Dex-P encapsulated in NPA coacervate to colitic SD rats showed significantly enhanced therapeutic outcomes than administering the equivalent amount of Dex-P in an aqueous solution.

### Example 12

### Regulation of Innate Immune Responses and Restoration of Gut Microbiota by Dex-P/NPA Treatment

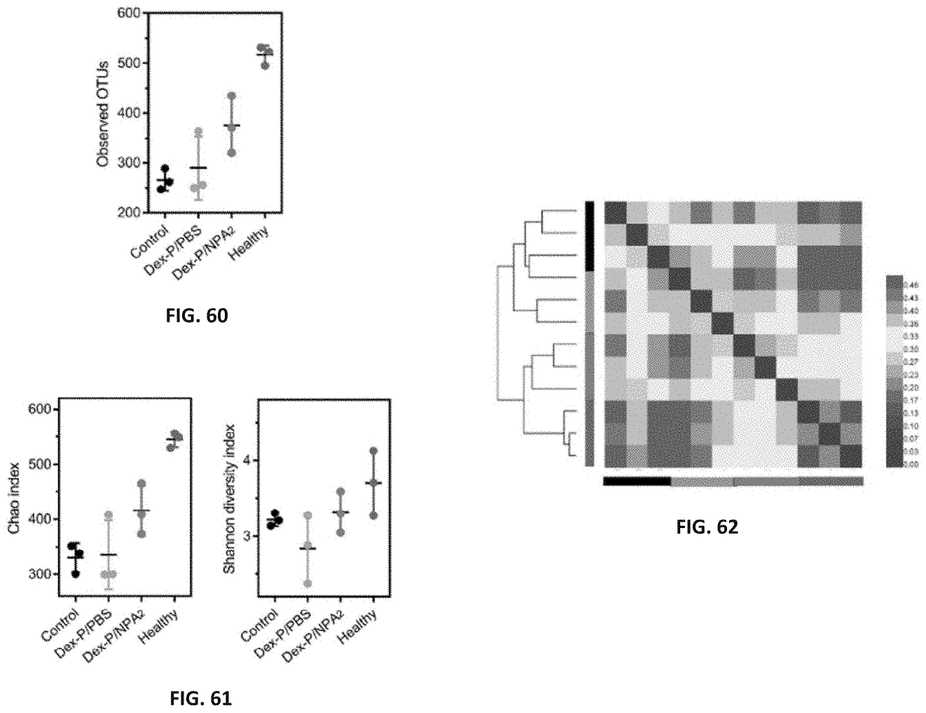

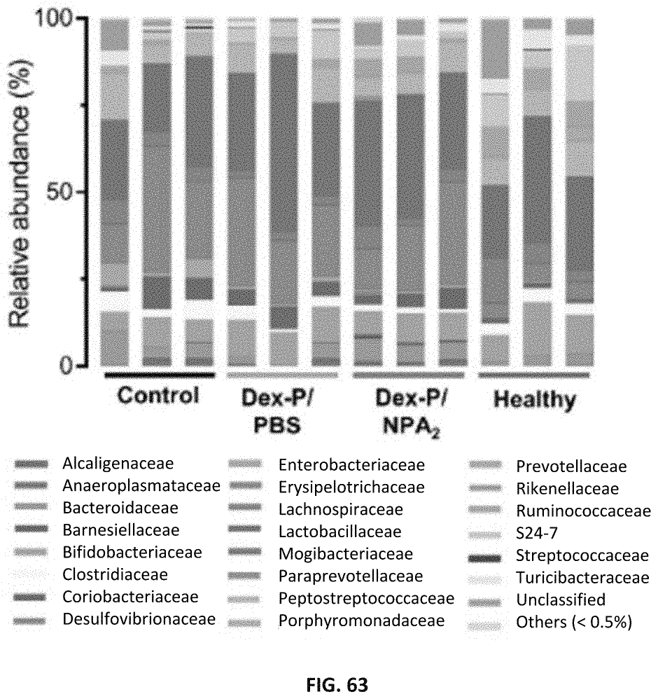

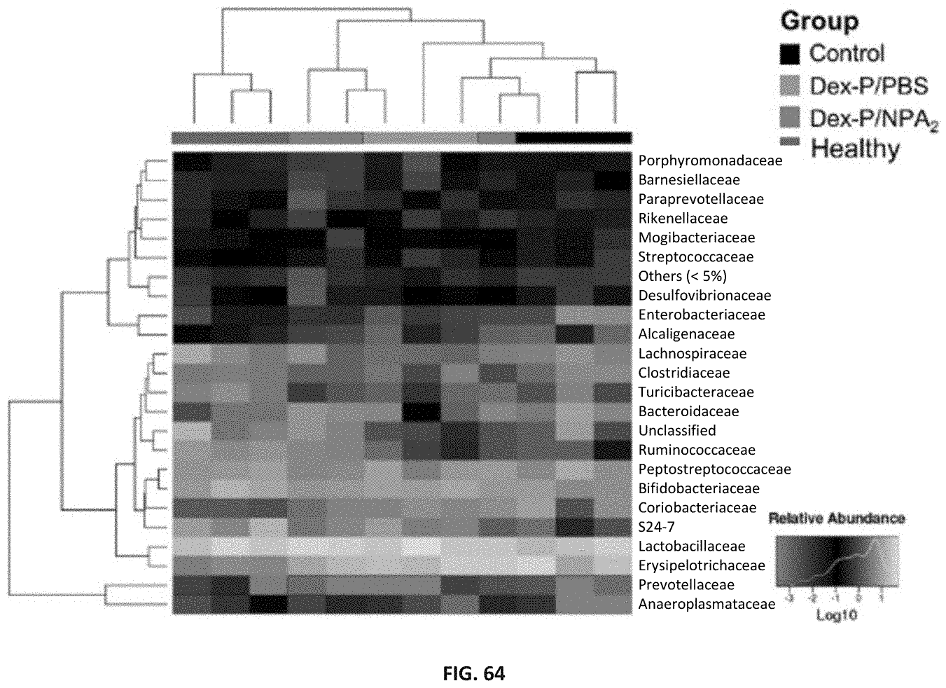

Analyses of fecal samples collected from randomly selected colitic SD rats on day 5 by sequencing the V4 region of the 16S ribosomal ribonucleic acid (rRNA) gene showed that Dex-P/NPA treatment indeed increased the bacterial richness (observed operational taxonomic units, OTUs) and diversity (Chao and Shannon indices) in colitic SD rats (FIGS. 60 and 61). In addition, a heatmap of β-diversity distance distribution was generated, and samples with similar β-diversity were clustered to reflect the similar compositions of gut microbiota (FIG. 62). The β-diversities between colitic SD rats receiving Dex-P/NPA and healthy SD rats were clustered more closely compared with colitic SD rats in the Dex-P/PBS group and the untreated control group (Control), thus suggesting the enhanced recovery of gut microbiota in colitic SD rats by Dex-P/NPA treatment. This was further confirmed by the taxonomic bacterial distribution histogram and clustered heatmap based on the relative abundance of gut microbiota at the family-level (FIGS. 63 and 64).

In summary, the examples provided herein demonstrate the development of physiologically stable NPA coacervate compartments, e.g., vacuoles or microdroplets, with low polydispersity to meet the stringent requirements for the formation of 3D compartmentalized cell microenvironments. The vacuolated NPA coacervate exhibits long-term resistance to coalescence under physiological condition and can restrict the diffusional exchange of macromolecules with surrounding liquid phase. The induced coacervate hydrogel immediately abolishes the diffusion barrier attributes of the NPA coacervate. We further demonstrated that the vacuolated NPA coacervate can control spatiotemporal distribution of macromolecules in the cell culture environment and therefore can regulate diverse functions of encapsulated cells. We believe that the NPA coacervate can become a new paradigm platform for the 3D culture of cells/organoids and drug delivery.

While the disclosure has been described in detail, modifications within the spirit and scope of the disclosure will be readily apparent to those of skill in the art in view of the foregoing discussion, relevant knowledge in the art and references discussed above in connection with the Background and Detailed Description, the disclosures of which are all incorporated herein by reference. In addition, it should be understood that aspects of the disclosure and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure.

1. A population of coacervates, each comprising an assembly of nanoparticles, wherein each nanoparticle comprises a hydrophobic core and a plurality of hydrophilic polymeric chains extending from the hydrophobic core, wherein the hydrophilic polymeric chains each comprise a functional end group, and wherein the coacervates further comprise non-covalent interactions between at least a portion of the functional end groups.

2. The coacervates of claim 1, wherein the nanoparticles each comprise an amphiphilic polymer, wherein the hydrophobic core comprises hydrophobic segments of the amphiphilic polymer, and wherein the hydrophilic polymeric chains comprise hydrophilic segments of the amphiphilic polymer.

3. The coacervates of claim 2, wherein the hydrophobic segments comprise alkyl groups.

4. The coacervates of claim 1, wherein the hydrophobic core comprises an inorganic material.

5. The coacervates of any claim 1, wherein the hydrophilic polymeric chains comprise a polyether.

6. The coacervates of claim 5, wherein the polyether comprises polyethylene glycol.

7. The coacervates of claim 1, wherein the functional end group comprises a hydroxylated aryl group.

8. The coacervates of claim 7, wherein the hydroxylated aryl group comprises a dihydroxybenzene.

9. The coacervates of claim 8, wherein the dihydroxybenzene comprises catechol.

10-18. (canceled)

19. A method of forming a population of coacervates, the method comprising:
providing a population of polymers, wherein each polymer comprises hydrophilic polymeric chains, and wherein each hydrophilic polymeric chain comprises a functional end group;
fabricating, via self-assembly of the polymers, a population of nanoparticles, wherein each nanoparticle comprises a hydrophobic core, and wherein each nanoparticle further comprises a plurality of the hydrophilic polymeric chains extending from the hydrophobic core; and
forming, via non-covalent interactions between at least a portion of the functional end groups, the population of coacervates.

20-28. (canceled)

29. The method of claim 19, wherein the forming comprises dialyzing a suspension of the population of nanoparticles against water.

30-32. (canceled)

33. A method of reversibly switching a physiological state of a population of coacervates, the method comprising:
providing the population of coacervates of claim 1, wherein the coacervates have a first physiological state; and
changing the temperature of the coacervates beyond an upper critical solution temperature, thereby switching the physiological state of the coacervates from the first physiological state to a second physiological state.

34. The method of claim 33, further comprising:
subsequent to the changing, adjusting the temperature of the coacervates beyond the upper critical solution temperature, thereby returning the physiological state of the coacervates from the second physiological state to the first physiological state.

35-37. (canceled)

38. A method of transitioning a population of coacervates from a vacuolated liquid state to a hydrogel state, the method comprising:
providing the population of coacervates of claim 1, wherein the coacervates have a vacuolated liquid state; and
contacting the coacervates with Ti⁴⁺ titanium ions, thereby transitioning the coacervates to a hydrogel state.

39. (canceled)

40. A method of transiently activating uptake of a macromolecule by a population of coacervates, the method comprising:
providing the population of coacervates of claim 1;
agitating the coacervates in a buffer comprising the macromolecule, thereby increasing the uptake efficiency of the coacervates and activating uptake of the macromolecule by the coacervates; and
stopping the agitation of the coacervates, thereby increasing the barrier efficiency of the coacervates.

41-42. (canceled)

43. A method of adhering a population of coacervates within the body of a subject, the method comprising:
providing the population of coacervates of claim 1, wherein the functional end group comprises catechol; and
administering the coacervates to the subject, thereby adhering the coacervates within the body of the subject.

44-45. (canceled)

46. A method of delivering a compound to a subject in need thereof, the method comprising:
providing the population of coacervates of claim 1, wherein the coacervates encapsulate a therapeutically effective amount of the compound; and
administering the coacervates to the subject, thereby delivering the compound.

47. A method of treating obesity, the method comprising:
administering to a subject in need thereof, a therapeutically effective amount of the population of coacervates of claim 1, wherein the functional end group comprises catechol, and wherein the coacervates adhere to the gastrointestinal tract of the subject.

48. A method for treating an inflammatory bowel disease, the method comprising:
providing the population of coacervates of claim 1, wherein the coacervates encapsulate a therapeutically effective amount of a compound for treating the inflammatory bowel disease, and wherein the functional end group comprises catechol; and
administering the coacervates to a subject, thereby adhering at least a portion of the coacervates to the gastrointestinal tract of the subject and delivering the compound.

49. A composition comprising:
the population of coacervates of claim 1; and
a pharmaceutically acceptable excipient.
